SEQUENCE LISTING (16 sequences)

SEQ ID NO: 1 - DNA, 23 bp, Artificial Sequence
  synthetic primer
cgactcactatagggagagcggc

SEQ ID NO: 2 - DNA, 24 bp, Artificial Sequence
  synthetic primer
aagaacatcgattttccatggcag

SEQ ID NO: 3 - DNA, 19 bp, Artificial Sequence
  synthetic primer
gggcaggaagagggcctat

SEQ ID NO: 4 - DNA, 17 bp, Artificial Sequence
  synthetic oligonucleotide
ggtgcagatgaacttca

SEQ ID NO: 5 - DNA, 19 bp, Artificial Sequence
  synthetic oligonucleotide
gctgattgctgtcacctgg

SEQ ID NO: 6 - DNA, 22 bp, Artificial Sequence
  synthetic oligonucleotide
gctgattgctgtcacctggagg

SEQ ID NO: 7 - DNA, 20 bp, Artificial Sequence
  synthetic primer
gtggcctggcactgagtaat

SEQ ID NO: 8 - DNA, 20 bp, Artificial Sequence
  synthetic primer
ctcagcaactctacaggcca

SEQ ID NO: 9 - DNA, 20 bp, Artificial Sequence
  synthetic primer
cactcattaggcaccccagg

SEQ ID NO: 10 - DNA, 20 bp, Artificial Sequence
  synthetic primer
cccaccctaccaaccaagtc

SEQ ID NO: 11 - DNA, 19 bp, Artificial Sequence
  synthetic oligonucleotide
gaagactactagccctgcc

SEQ ID NO: 12 - DNA, 23 bp, Artificial Sequence
  synthetic oligonucleotide
ggaagactactagccctgccagg

SEQ ID NO: 13 - DNA, 20 bp, Artificial Sequence
  synthetic primer
ccagcctgcaatctgctttt

SEQ ID NO: 14 - DNA, 20 bp, Artificial Sequence
  synthetic primer
gccaactgcagggacttcta

SEQ ID NO: 15 - DNA, 20 bp, Artificial Sequence
  synthetic primer
gaccaggagtgctctatggc

SEQ ID NO: 16 - DNA, 25 bp, Artificial Sequence
  synthetic primer
aggaaatagagagaatccgaatctc

(19) United States
(12) Patent Application Publication
(10) Pub. No.: US 2019/0225989 A1
(43) Pub. Date: Jul. 25, 2019

(54) GENE KNOCKIN METHOD AND KIT FOR GENE KNOCKIN

(71) Applicant: Institute of Hematology and Blood Disease Hospital, CAMS & PUMC, Tianjin (CN)

(72) Inventors: Tao CHENG, Tianjin (CN); Jianping ZHANG, Tianjin (CN); Xiaolan LI, Tianjin (CN); Xiao-Bing ZHANG, Loma Linda, CA (US)

(73) Assignee: Institute of Hematology and Blood Disease Hospital, CAMS & PUMC, Tianjin (CN)

(21) Appl. No.: 15/874,904

(22) Filed: Jan. 19, 2018

(51) Int. Cl. C12N 15/90; C12N 15/11; C12N 9/22; C12N 5/0783

(52) U.S. Cl. CPC C12N 15/907; C12N 15/11; C12N 9/22; C12N 2501/405; C12N 2310/20; C12N 2800/80; C12N 5/0636

(57) ABSTRACT

A gene knockin method and a kit for gene knockin are provided. The method comprises (a) introducing a RNA-guided endonuclease that cleaves the chromosome at the insertion site into the cell; (b) introducing a guide RNA into the cell; and (c) introducing a donor plasmid into the cell, wherein the donor plasmid comprises the donor sequence flanked with a 5′ homology arm and a 3′ homology arm, a 5′ flanking sequence upstream of the 5′ homology arm, and a 3′ flanking sequence downstream of the 3′ homology arm, wherein the 5′ homology arm is homologous to a 5′ target sequence upstream of the insertion site on the genome and the 3′ homology arm is homologous to a 3′ target sequence downstream of the insertion site on the genome, wherein the guide RNA recognizes the insertion site, the 5′ flanking sequence, and the 3′ flanking sequence, wherein the RNA-guided endonuclease cleaves the donor plasmid at the 5′ flanking sequence and the 3′ flanking sequence, thereby producing a linear nucleic acid, wherein the donor sequence is inserted in to the genome at the insertion site through homology-directed repair.

### BACKGROUND

### Technical Field

The invention relates to a method for genome editing and a kit for genome editing, and more particularly, to a gene knockin method and a kit for gene knockin.

### Description of Related Art

The ability to precisely edit genomes endows scientists with a powerful tool to interrogate the functionalities of any pieces of DNA in the genome of any species and it may also lead to the development of new therapies that can potentially cure numerous genetic diseases. However, precise gene editing by homologous recombination is very inefficient, unless a DNA double-stranded break (DSB) is created at the targeting site, which increases homology-directed repair (HDR) mediated gene editing efficiency by ˜1000-fold. To induce DSB at a desired site, several technologies have been developed over the past decade, including zinc-finger nucleases (ZFN), transcription activator-like effector nucleases (TALEN), and the clustered regularly interspaced short palindromic repeats (CRISPR)/CRISPR-associated protein-9 nuclease (Cas9) system. The CRISPR-Cas9 system has caught widespread attention due to its robust performance, simple vector construction, and multiplexability in manipulating genes.

CRISPR is an adoptive immune system evolved in bacteria and archaea to fight against invading agents such as bacteriophages or plasmids. Diverse CRISPR systems have been adapted for use in editing mammalian genomes. Currently the most commonly used system is derived from Streptococcus pyogenes (Sp), which consists of a Cas9 endonuclease and two separate small RNAs, called tracrRNA (trans-activating crRNA) and crRNA (clustered regularly interspaced short palindromic repeats RNA, CRISPR RNA), that can be combined with a tetraloop to form a single guide RNA (sgRNA). SpCas9, which will be referred to henceforth as Cas9 for simplicity, cuts double strands of DNA to generate blunt-ended double strand breaks (DSBs) at 3 bp upstream of the NGG PAM (protospacer adjacent motif) under the guidance of sgRNA, which specifically recognizes the chromosomal loci of interest with 17-20 nucleotides (nt). Cells repair DSBs primarily by two mechanisms: non-homologous end joining (NHEJ) and homology-directed repair (HDR). In comparison to NHEJ, which generates a knockout phenotype by introducing variable insertions or deletions (indels) at the DSB, the HDR pathway creates precise deletions, base substitution, or insertion of coding sequences of interest in the presence of a recombination donor flanked with right and left homology arms (HA). Thus, the HDR pathway can be exploited to facilitate correction of diseased genes, insertion of epitope tags or fluorescent reporters, and overexpression of genes of interest in a site-specific manner.

Using rationally designed sgRNAs, high-level gene knockout can be achieved in different types of cells. However, improving the efficiency of precise CRISPR/Cas9-mediated gene editing or HDR-mediated knockin (KI) remains a major challenge, especially in human induced pluripotent stem cells (iPSCs). Significant effort has been devoted to increasing knockin efficiency by improving targeting strategies, especially for insertion of a large DNA fragment. Previous reports used ZFN, TALEN, or CRISPR-Cas9 technology to knock in long DNA fragments via a homology-independent manner. In these methods, the donor plasmid contains an endonuclease cleavage site and can be linearized in vivo when co-transfected with a specific endonuclease. While these approaches are generic, they often lead to the integration of the entire donor plasmid and may induce mutagenic junctions caused by erroneous NHEJ, limiting the application potentials, and therefore the development of a novel method for efficient precise gene knockin is an important current object.

### SUMMARY

The invention provides a method for efficient precise gene knockin.

The invention provides a kit for efficient precise gene knockin.

In one embodiment, disclosed herein is a method of inserting a donor sequence at a predetermined insertion site on a genome in an eukaryotic cell, comprising: introducing a RNA-guided endonuclease, a guide RNA and a donor plasmid, wherein the donor plasmid comprises the donor sequence flanked with a 5′ homology arm and a 3′ homology arm, a 5′ flanking sequence upstream of the 5′ homology arm, and a 3′ flanking sequence downstream of the 3′ homology arm, wherein the 5′ homology arm is homologous to a 5′ target sequence upstream of the insertion site on the genome and the 3′ homology arm is homologous to a 3′ target sequence downstream of the insertion site on the genome, wherein the guide RNA recognizes the insertion site, the 5′ flanking sequence, and the 3′ flanking sequence, wherein the RNA-guided endonuclease cleaves the genome at the insertion site, wherein the RNA-guided endonuclease cleaves the donor plasmid at the 5′ flanking sequence and the 3′ flanking sequence to produce a linear nucleic acid, and wherein the donor sequence is inserted in to the genome at the insertion site through homology-directed repair.

In some embodiments, the 5′ homology arm and the 3′ homology arm may be at least about 50 bp in length, respectively.

In some embodiments, the 5′ homology arm and the 3′ homology arm may range from about 50 bp to about 2000 bp in length, respectively.

In some embodiments, the 5′ target sequence and the 3′ target sequence may be less than 200 bp away from the insertion site.

In some embodiments, the 5′ target sequence and the 3′ target sequence may be separated by less than 200 bp.

In some embodiments, the method may further comprise introducing a cell cycle regulator into the cell.

In some embodiments, the cell cycle regulator may comprise CCND1, Nocodazole, or a combination thereof.

In some embodiments, the RNA-guided endonuclease may be Cas9.

In some embodiments, the guide RNA may comprise a CRISPR RNA (crRNA) and a tracrRNA.

In some embodiments, the cell cycle regulator may be introduced into the cell in the form of a protein, a mRNA, or a cDNA.

In some embodiments, the eukaryotic cell may be a mammalian cell.

In some embodiments, the eukaryotic cell may comprise a pluripotent stem cell or an adult stem cell.

In one embodiment, disclosed herein is a kit for inserting a donor sequence at a predetermined insertion site on a genome in an eukaryotic cell, comprising: a RNA-guided endonuclease; a guide RNA; and a donor plasmid, wherein the donor plasmid comprises the donor sequence flanked with a 5′ homology arm and a 3′ homology arm, a 5′ flanking sequence upstream of the 5′ homology arm, and a 3′ flanking sequence downstream of the 3′ homology arm, wherein the 5′ homology arm is homologous to a 5′ target sequence upstream of the insertion site on the genome and the 3′ homology arm is homologous to a 3′ target sequence downstream of the insertion site on the genome, wherein the guide RNA is able to recognize the insertion site, the 5′flanking sequence, and the 3′flanking sequence, wherein the RNA-guided endonuclease is able to cleave the chromosome at the insertion site, wherein the donor plasmid is cleaved at the 5′ flanking sequence and the 3′ flanking sequence within the cell to produce a linear nucleic acid.

In some embodiments, the 5′ homology arm and the 3′ homology arm may be at least about 50 bp in length, respectively.

In some embodiments, the 5′ homology arm and the 3′ homology arm may range from about 50 bp to about 2000 bp in length, respectively.

In some embodiments, the kit may further comprise a cell cycle regulator.

In some embodiments, the cell cycle regulator may comprise CCND1, Nocodazole, or a combination thereof.

In some embodiments, the RNA-guided endonuclease may be Cas9.

In some embodiments, the guide RNA may comprise a CRISPR RNA (crRNA) and a tracrRNA.

### BRIEF DESCRIPTION OF THE DRAWINGS

FIG. 1 depicts a scheme of mCherry HDR reporter system.

FIG. 2 depicts a scheme design of pD-mCherry donor and pD-mCherry-sg donor.

FIG. 3A and FIG. 3B depict flow cytometry analysis of 293 T reporter cell after co-transfection of Cas9/conventional pD-mCherry donor, compared to Cas9/pD-mCherry-sg donor.

FIG. 4 depicts a schematic design of pD-mCherry-sg donor with HA in the range of 0˜1500 bp in length.

FIG. 5A and FIG. 5B depict flow cytometry analysis of 293 T reporter cell after co-transfection of Cas9/conventional pD-mCherry donor, compared to Cas9/pD-mCherry-sg donor.

FIG. 6 depicts a scheme of genome editing at the CTNNB1 locus in iPSCs.

FIG. 7A and FIG. 7B depict flow cytometry analysis of human iPSCs after co-transfection of Cas9/conventional pD-mNeonGreen donor, compared to pD-mNeonGreen-sg donor.

FIG. 8 depicts a scheme of different knockin patterns.

FIG. 9 shows a result of PCR analysis for knockin pattern.

FIG. 10 shows a distribution of different knockin patterns by double cut HDR donors with different HA lengths.

FIG. 11 shows a quantitative PCR (qPCR) analysis of donor plasmid backbone-forward insertion.

FIG. 12A depicts a schematic illustration of the replaced sequence (RS) in pD-mNEonGreen-sg-RS1-39 bp-HA300-300 bp donor.

FIG. 12B depicts a schematic illustration of the replaced sequence (RS) in pD-mNEonGreen-sg-RS1-0 bp-HA300-300 bp.

FIG. 13 depict flow cytometry analysis of human iPSCs after co-transfection of Cas9/pD-mNEonGreen-sg-RS1-0 bp-HA300-300 bp donor, compared to pD-mNeonGreen-sg-RS1-39 bp-HA300-300 bp donor.

FIG. 14 shows a distribution of different knockin patterns when using two donors.

FIG. 15 shows a quantitative PCR (qPCR) analysis of donor plasmid backbone-forward insertion.

FIG. 16 depicts a scheme of genome editing at the PRDM14 locus in iPSCs. PRDM14 is a regulator of pluripotency.

FIG. 17A and FIG. 17B depict flow cytometry analysis of human iPSCs after co-transfection of Cas9/conventional pD-GFP donor, compared to pD-GFP-sg donor.

FIG. 18 depicts a scheme of different knockin patterns.

FIG. 19 is a result of PCR analysis for knockin pattern.

FIG. 20 shows a distribution of different knockin patterns by double cut HDR donors with different HA lengths.

FIG. 21 shows the effects of small molecules on HDR efficiency at the CTNNB1 or PRDM14 locus in iPSCs.

FIG. 22 shows the effects of small molecules on HDR efficiency at the CTNNB1 or PRDM14 locus in the H1 ES cells.

FIG. 23 shows the effects of RAD51, Ad4E1B-Eorf46, and CCND1 on HDR efficiency at the CTNNB1 or PRDM14 locus in iPSCs.

FIG. 24 shows the effects of Nocodazole and CCND1 on HDR efficiency at the CTNNB1 or PRDM14 locus in iPSCs.

FIG. 25 shows a distribution of different knockin patterns by CCND1.

### DESCRIPTION OF THE EMBODIMENTS

The present invention provides a novel DNA knock-in method which allows for the introduction of one or more exogenous sequences into a specific target site on the cellular chromosome with significantly higher efficiency compared to traditional DNA knock-in methods using RNA-guided endonuclease such as CRISPR/Cas9 or TALEN-based gene knock-in systems. In addition to the use of a RNA-guided endonuclease, the method of the present application further utilizes a donor plasmid which comprises a 5′ flanking sequence upstream of the 5′ homology arm and a 3′ flanking sequence downstream of the 3′ homology arm. The RNA-guided endonuclease cleaves the donor plasmid at both of the flanking sequences, thereby producing the linear nucleic acid. The gene knockin system allows donor sequences to be inserted at any desired target site with high efficiency, making it feasible for many uses such as creation of transgenic animals expressing exogenous genes, modifying (e.g., mutating) a genomic locus, and gene editing, for example by adding an exogenous non-coding sequence (such as sequence tags or regulatory elements) into the genome. The improved gene knockin system is broadly applicable in generating precise knockin or reporter animals and human cell lines for basic research and disease modeling. Further improvements of the gene knockin system may contribute to the success of next-generation clinical gene therapy.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

The term “sequence” refers to a nucleotide sequence of any length, which can be DNA or RNA; can be linear, circular or branched and can be either single-stranded or double stranded.

The term “homologous nucleic acid” as used herein includes a nucleic acid sequence that is either identical or substantially similar to a known reference sequence. In one embodiment, the term “homologous nucleic acid” is used to characterize a sequence that is at least 70%, at least 75%, at least 80%, at least 85%, at least 90%, at least 95%, at least 96%, at least 97%, at least 98%, at least 99%, or even 100% identical to a known reference sequence.

The term “homology-directed repair (HDR)” refers to the specialized form DNA repair that takes place, for example, during repair of double-strand breaks in cells. This process requires nucleotide sequence homology, uses a “donor” molecule to template repair of a “target” molecule (i.e., the one that experienced the double-strand break), and leads to the transfer of genetic information from the donor to the target. Homology-directed repair may result in an alteration of the sequence of the target molecule (e.g., insertion, deletion, mutation), if the donor polynucleotide differs from the target molecule and part or all of the sequence of the donor polynucleotide is incorporated into the target DNA.

The term “non-homologous end joining (NHEJ)” refers to the repair of double-strand breaks in DNA by direct ligation of the break ends to one another without the need for a homologous template (in contrast to homology-directed repair, which requires a homologous sequence to guide repair). NHEJ often results in the loss (deletion) of nucleotide sequence near the site of the double-strand break.

The term “induced pluripotent stem cell” or “iPSC” refers to a PSC that is derived from a cell that is not a PSC {i.e., from a cell this is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei.

The term “donor sequence” as used herein refers to a nucleic acid to be inserted into the chromosome of a host cell. A donor sequence can be of any length, for example between 2 and 10,000 nucleotides in length (or any integer value therebetween or thereabove).

Reference to “about” a value or parameter herein includes (and describes) variations that are directed to that value or parameter per se. For example, description referring to “about X” includes description of “X”.

As used herein and in the appended claims, the singular forms “a”, “or”, and “the” include plural referents unless the context clearly dictates otherwise.

The compositions and methods of the present invention may comprise, consist of, or consist essentially of the essential elements and limitations of the invention described herein, as well as any additional or optional ingredients, components, or limitations described herein or otherwise useful in a nutritional or pharmaceutical application.

The present invention provides methods of inserting a donor sequence at a predetermined insertion site on a genome in an eukaryotic cell. In some embodiments, the method comprises: introducing a RNA-guided endonuclease, a guide RNA and a donor plasmid into the cell, wherein the donor plasmid comprises the donor sequence flanked with a 5′ homology arm and a 3′ homology arm, a 5′ flanking sequence upstream of the 5′ homology arm, and a 3′ flanking sequence downstream of the 3′ homology arm, wherein the 5′ homology arm is homologous to a 5′ target sequence upstream of the insertion site on the genome and the 3′ homology arm is homologous to a 3′ target sequence downstream of the insertion site on the genome, wherein the guide RNA recognizes the insertion site, the 5′ flanking sequence, and the 3 ‘flanking sequence, wherein the RNA-guided endonuclease cleaves the genome at the insertion site, wherein the RNA-guided endonuclease cleaves the donor plasmid at the 5’ flanking sequence and the 3′ flanking sequence to produce a linear nucleic acid, and wherein the donor sequence is inserted in to the genome at the insertion site through homology-directed repair.

In some embodiments, the cells described herein may be any eukaryotic cell, e.g., an isolated cell of an animal, such as a totipotent, pluripotent, or adult stem cell, a zygote, or a somatic cell. In some embodiments, the eukaryotic cell is mammalian cell. In some embodiments, the eukaryotic cell is from a primary cell culture. In some embodiments, eukaryotic cells for use in the methods are human cells. In some embodiments, the eukaryotic cell comprises a pluripotent stem cell or an adult stem cell. In some embodiments, the eukaryotic cells may be human cells, which include, but are not limited to human induced pluripotent stem cells (iPSCs) and 293T (or HEK293T) cell.

In some embodiments, the RNA-guided endonuclease, the guide RNA, and the donor plasmid are introduced into the cell simultaneously. In some embodiments, at least one of the three components is introduced into the cell at a different time from the other components. For example, the donor plasmid may be introduced into the cell first, and the RNA-guided endonuclease and the guide RNA are subsequently introduced. In some embodiments, the RNA-guided endonuclease is introduced into the cell first, and the donor plasmid and the guide RNA are subsequently introduced. In some embodiments, all three components are introduced at a different time point relative to each other. For example, the three components can be administered in a sequence, one after another at a specific order.

In some embodiments, the RNA-guided endonuclease is introduced into the eukaryotic cell in the form of a protein, or in the form of a nucleic acid encoding the RNA-guided endonuclease, such as a messenger RNA (mRNA), or a cDNA. Nucleic acids can be delivered as part of a larger construct, such as a plasmid or viral vector, or directly, e.g., by electroporation, lipid vesicles, viral transporters, and microinjection. For example, the RNA-guided endonuclease can be introduced into the cell by a variety of means known in the art, including transfection, calcium phosphate-DNA co-precipitation, DEAE-dextran-mediated transfection, polybrene-mediated transfection, electroporation, microinjection, transduction, cell fusion, liposome fusion, lipofection, protoplast fusion, retroviral infection, use of a gene gun, use of a DNA vector transporter, and biolistics (e.g., particle bombardment).

In some embodiments, the nucleic acid encoding the RNA-guided endonuclease is introduced into the cell by transfection (including for example transfection through electroporation). In some embodiments, the nucleic acid encoding the RNA-guided endonuclease is introduced into the cell by injection.

In some embodiments, the guide RNA (gRNA) can be introduced, for example, as RNA or as a plasmid or other nucleic acid vector encoding the guide RNA. The RNA-guided endonuclease binds to the gRNA and the target DNA to which the gRNA binds and cleaves the chromosome at the insertion site. For example, the guide RNA (gRNA) can be introduced into the cell by a variety of means known in the art, including transfection, calcium phosphate-DNA co-precipitation, DEAE-dextran-mediated transfection, polybrene-mediated transfection, electroporation, microinjection, transduction, cell fusion, liposome fusion, lipofection, protoplast fusion, retroviral infection, use of a gene gun, use of a DNA vector transporter, and biolistics (e.g., particle bombardment).

The introduced donor plasmid introduced may be cleaved within the cell to produce a linear nucleic acid. It can be delivered by any method appropriate for introducing nucleic acids into a cell. For example, the donor plasmid can be introduced into the cell by a variety of means known in the art, including transfection, calcium phosphate-DNA co-precipitation, DEAE-dextran-mediated transfection, polybrene-mediated transfection, electroporation, microinjection, transduction, cell fusion, liposome fusion, lipofection, protoplast fusion, retroviral infection, use of a gene gun, use of a DNA vector transporter, and biolistics (e.g., particle bombardment).

In some embodiments, the RNA-guided endonuclease is a sequence-specific nuclease. The term “sequence-specific nuclease,” as used herein, refers to a protein that recognizes and binds to a polynucleotide at a specific nucleic acid sequence and catalyzes a double-strand break in the polynucleotide. In certain embodiments, the RNA-guided endonuclease cleaves the chromosome only once, i.e., a single double-strand break is introduced at the insertion site during the methods described herein.

An example of a RNA-guided endonuclease system that can be used with the methods and compositions described herein includes the Cas/CRISPR system. The Cas/CRISPR (Clustered Regularly interspaced Short Palindromic Repeats) system exploits RNA-guided DNA-binding and sequence-specific cleavage of target DNA. A guide RNA (gRNA) contains about 20-25 (such as 20) nucleotides that are complementary to a target genomic DNA sequence upstream of a genomic PAM (protospacer adjacent motifs) site and a constant RNA scaffold region. In certain embodiments, the target sequence is associated with a PAM, which is a short sequence recognized by the CRISPR complex. The precise sequence and length requirements for the PAM differ depending on the CRISPR enzyme used, but PAMs are typically 2-5 bp sequences adjacent to the protospacer (that is, the target sequence). Examples of PAM sequences are known in the art, and the skilled person will be able to identify further PAM sequences for use with a given CRISPR enzyme. For example, target sites for Cas9 from S. pyogenes, with PAM sequences NGG, may be identified by searching for 5′-Nx-NGG-3′ both on an input sequence and on the reverse-complement of the input. In certain embodiments, the genomic PAM site used herein is NGG, NNG, NAG, NGGNG, or NNAGAAW. In particular embodiments, the Streptococcus pyogenes Cas9 (SpCas9) is used and the corresponding PAM is NGG. In some aspects, different Cas9 enzymes from different bacterial strains use different PAM sequences. The Cas (CRISPR-associated) protein binds to the gRNA and the target DNA to which the gRNA binds and introduces a double-strand break in a defined location upstream of the PAM site. In one aspect, the CRISPR/Cas, Cas/CRISPR, or the CRISPR-Cas system (these terms are used interchangeably throughout this application) does not require the generation of customized proteins to target specific sequences but rather a single Cas enzyme can be programmed by a short RNA molecule to recognize a specific DNA target, i.e., the Cas enzyme can be recruited to a specific DNA target using the short RNA molecule.

In some embodiments, the RNA-guided endonuclease is a type II Cas protein. In some embodiments, the RNA-guided endonuclease is Cas9, a homolog thereof, or a modified version thereof. In some embodiments, a combination of two or more Cas proteins can be used. In some embodiments, the CRISPR enzyme is Cas9, and may be Cas9 from S. pyogenes or S. pneumoniae. In some embodiments, Cas9 is used in the methods described herein. Cas9 harbors two independent nuclease domains homologous to HNH and RuvC endonucleases, and can cut double strands of DNA to generate blunt-ended double strand breaks (DSBs) under the guidance of gRNA.

In some embodiments, a guide RNA is an RNA comprising a 5′ region comprising at least one repeat from a CRISPR locus and a 3′ region that is complementary to the predetermined insertion site on the chromosome. In certain embodiments, the 5′ region comprises a sequence that is complementary to the predetermined insertion site on the chromosome, and the 3′ region comprises at least one repeat from a CRISPR locus. In some aspects, the 3′ region of the guide RNA further comprises the one or more structural sequences of crRNA and/or tracrRNA. In some embodiments, the guide RNA comprises a crRNA and a tracrRNA, and the two pieces of RNA form a complex through hybridization.

In some embodiments, the insertion of the donor sequence can be evaluated using any methods known in the art. For example, a 5′ primer corresponding to a sequence upstream of the 5′ homology arm and a corresponding 3′ primer corresponding to a region in the donor sequence can be designed to assess the 5′-junction of the insertion. Similarly, a 3′ primer corresponding to a sequence downstream of the 3′ homology arm and a corresponding 5′ primer corresponding to a region in the donor sequence can be designed to assess the 3′ junction of the insertion.

In some embodiments, the insertion site can be at any desired site, so long as RNA-guided endonuclease can be designed to effect cleavage at such site. In some embodiments, the insertion site is at a target gene locus. In some embodiments, the insertion site is not a gene locus.

In some embodiments, the donor nucleic acid is a sequence not present in the host cell. In some embodiments, the donor sequence is an endogenous sequence present at a site other than the predetermined target site. In some embodiments, the donor sequence is a coding sequence. In some embodiments, the donor sequence is a non-coding sequence. In some embodiments, the donor sequence is a mutant locus of a gene.

In some embodiments, the size of the donor sequence can range from about 1 bp to about 100 kb. In certain embodiments, the size of the donor sequence is between about 1 bp and about 10 bp, between about 10 bp and about 50 bp, between about 50 bp and about 100 bp, between about 100 bp and about 500 bp, between about 500 bp and about 1 kb, between about 1 kb and about 10 kb, between about 10 kb and about 50 kb, between about 50 kb and about 100 kb, or more than about 100 kb.

In some embodiments, the donor sequence is an exogenous gene to be inserted into the chromosome. In some embodiments, the donor sequence is modified sequence that replaces the endogenous sequence at the target site. For example, the donor sequence may be a gene harboring a desired mutation, and can be used to replace the endogenous gene present on the chromosome. In some embodiments, the donor sequence is a regulatory element. In some embodiments, the donor sequence is a tag or a coding sequence encoding a reporter protein and/or RNA. In some embodiments, the donor sequence is inserted in frame into the coding sequence of a target gene which will allow expression of a fusion protein comprising an exogenous sequence fused to the N- or C-terminus of the target protein.

In some embodiments, the donor plasmid described herein is cleaved within the cell to produce a linear nucleic acid. The linear nucleic acid described herein comprises a 5′ homology arm, a donor sequence, and a 3′ homology arm. In other words, the donor sequence is flanked with a 5′ homology arm and a 3′ homology arm.

In some embodiments, the homology anus are at least about 50 bp in length, for example at least about any of 50 bp, 100 bp, 200 bp, 300 bp, 600 bp, 900 bp, 1 kb, 1.5 kb, 2 kb, 4 kb, 6 kb, 10 kb, 15 kb and 20 kp in length. In some embodiments, the homology arms are at least about 300 bp in length. In certain embodiments, the homology arms may range from about 50 bp to about 2000 bp, from about 100 bp to about 2000 bp, from about 150 bp to about 2000 bp, from about 300 bp to about 2000 bp, from about 300 bp to about 1500 bp, from about 300 bp to about 1000 bp in length. In some embodiments, the length of the 5′ homology arm and the length of the 3′ homology arm are the same. In some embodiments, the length of the 5′ homology arm is different from that of the 3′ homology arm.

In some embodiments, the 5′ homology arm is homologous to a 5′ target sequence upstream of the insertion site on the genome and the 3′ homology arm is homologous to a 3′ target sequence downstream of the insertion site (e.g. DSB) on the genome, thereby allowing homology-directed repair to occur. In some embodiments, the 5′ and/or 3′ homology arms may be homologous to corresponding target sequences that is less than 200 bp away from the insertion site (e.g. DNA cleavage site). In some embodiments, the 5′ and/or 3′ homology arms may be homologous to a target sequence that is 0 bp away from the DNA cleavage site. In some embodiments, the 5′ target sequence and the 3′ target sequence may be separated by less than 200 bp.

In some embodiments, the donor plasmid is cleaved within the cell (for example by a RNA-guided endonuclease recognizing a cleavage site on the plasmid) to produce a linear nucleic acid described herein. For example, the donor plasmid may comprise flanking sequences upstream of the 5′ homology arm and downstream of the 3′ homology alai. Such flanking sequences in some embodiments do not exist in the genomic sequences of the host cell thus allowing cleavage to only occur on the donor plasmid. The guide RNA recognizes the 5′ flanking sequence and the 3′ flanking sequence. RNA-guided endonuclease can then be designed accordingly to effect cleavage at the 5′ flanking sequence and the 3′ flanking sequence under the guidance of guide RNA that allows the release of the linear nucleic acid without affecting the host sequences. The flanking sequences can be, for example, about 20 to about 23 bp.

In some embodiments, the method further comprises introducing a cell cycle regulator into the cell. In some embodiments, the cell cycle regulator is introduced into the eukaryotic cell in the form of a protein, or in the faun of a nucleic acid encoding the cell cycle regulator, such as a messenger RNA (mRNA), or a cDNA. Nucleic acids can be delivered as part of a larger construct, such as a plasmid or viral vector, or directly, e.g., by electroporation, lipid vesicles, viral transporters, and microinjection. In some embodiments, the cell cycle regulator may be introduced into the cell by a variety of means known in the art, including transfection, calcium phosphate-DNA co-precipitation, DEAE-dextran-mediated transfection, polybrene-mediated transfection, electroporation, microinjection, transduction, cell fusion, liposome fusion, lipofection, protoplast fusion, retroviral infection, use of a gene gun, use of a DNA vector transporter, and biolistics (e.g., particle bombardment). In some embodiments, the cell cycle regulator is directly added into medium and then is introduced into the cell by contacting the cell with the cell cycle regulator. In some embodiments, at least two cell cycle regulators are introduced into the cell. In some embodiments, two cell cycle regulators are introduced into the cell, wherein one of the regulators is introduced into the eukaryotic cell in the form of a nucleic acid encoding the cell cycle regulator and the other cell cycle regulator is introduced in the form of a protein.

In some embodiments, the RNA-guided endonuclease, the guide RNA, the donor plasmid, and the cell cycle regulator are introduced into the cell simultaneously. In some embodiments, at least one of the four components is introduced into the cell at a different time from the other components. For example, the donor plasmid may be introduced into the cell first, and the RNA-guided endonuclease, the guide RNA, and the cell cycle regulator are subsequently introduced. In some embodiments, the RNA-guided endonuclease is introduced into the cell first, and the donor plasmid, the guide RNA, and the cell cycle regulator are subsequently introduced. In some embodiments, all four components are introduced at a different time point relative to each other. For example, the four components can be administered in a sequence, one after another at a specific order. In some embodiments, the RNA-guided endonuclease, the donor plasmid, and the guide RNA are introduced into the cell first, and the cell cycle regulator is subsequently introduced.

In some embodiments, the cell cycle regulator is constitutively overexpressed in the cell. In some embodiments, the cell cycle regulator is transiently overexpressed. For example, in some cases, the cell cycle regulator is overexpressed for a period of time of from about 1 hour to about 36 hours. In some cases, the cell cycle regulator is overexpressed for a period of time of from about 1 hour to about 48 hours. For example, in some cases, the cell cycle regulator is overexpressed for a period of time of from about 1 hour to about 4 hours, from about 4 hours to about 8 hours, from about 8 hours to about 12 hours, from about 12 hours to about 16 hours, from about 16 hours to about 20 hours, from about 20 hours to about 24 hours, from about 24 hours to about 30 hours, from about 30 hours to about 36 hours, from about 36 hours to about 42 hours, or from about 42 hours to about 48 hours. In some cases, the cell cycle regulator is overexpressed for a period of time of from about 1 hour to about 72 hours. In some cases, the cell cycle regulator is overexpressed for a period of time of from about 48 hours to about 72 hours.

In some embodiments, the cell cycle regulator is introduced in to the cell such that the level of the cell cycle regulator in the cell is at least 20%, at least 50%, at least 75%, at least 1-fold, at least 2-fold, or more than 2-fold, higher than the background level of the cell cycle regulator in a control (unmodified) cell. For example, in some cases, the cell cycle regulator is introduced in to the cell such that the level of the cell cycle regulator in the cell is from 25% to 50%, from 50% to 75%, from 75% to 1-fold, or from 1-fold to 2-fold, higher than the background level of the cell cycle regulator in a control (unmodified) cell.

In some embodiments, the cell cycle regulator comprises CCND1, Nocodazole, or a combination thereof. In some embodiments, the cell cycle regulator is CCND1 and is introduced into the cell in the form of in the form of a protein. In some embodiments, the cell cycle regulator is Nocodazole and is introduced into the cell in the form of a nucleic acid encoding the cell cycle regulator.

Also provided herein are kits useful for any one of the methods described herein. For example, in some embodiments, there is provided a kit for inserting a donor sequence at a predetermined insertion site on a genome in an eukaryotic cell, comprising: (a) a RNA-guided endonuclease that cleaves the chromosome at the insertion site into the cell; (b) a guide RNA; and (c) a donor plasmid, wherein the donor plasmid comprises the donor sequence flanked with a 5′ homology arm and a 3′ homology arm, a 5′ flanking sequence upstream of the 5′ homology arm, and a 3′ flanking sequence downstream of the 3′ homology arm, wherein the 5′ homology arm is homologous to a 5′ target sequence upstream of the insertion site on the genome and the 3′ homology arm is homologous to a 3′ target sequence downstream of the insertion site on the genome, wherein the guide RNA recognizes the insertion site, the 5′flanking sequence, and the 3′flanking sequence, wherein the donor plasmid can be cleaved at the 5′ flanking sequence and the 3′ flanking sequence within the cell to produce a linear nucleic acid.

In some embodiments, the kit further comprises a cell cycle regulator. The cell cycle regulator comprises CCND1, Nocodazole, or a combination thereof.

The kits described herein may also comprise a packaging to house the contents of the kit. The packaging optionally provides a sterile, contaminant-free environment, and can be made of any of plastic, paper, foil, glass, and the like. In some embodiments, the packaging is a glass vial. In some embodiments, the kit further comprises an instruction for carrying out any one of the methods described herein.

In the following, the above embodiments are described in more detail with reference to examples. However, the examples are not to be construed as limiting the scope of the invention in any sense.

### Example 1: A Double Cut HDR Donor Increases HDR Efficiency in 293 T Cells

[Establishment of mCherry HDR Reporter System]

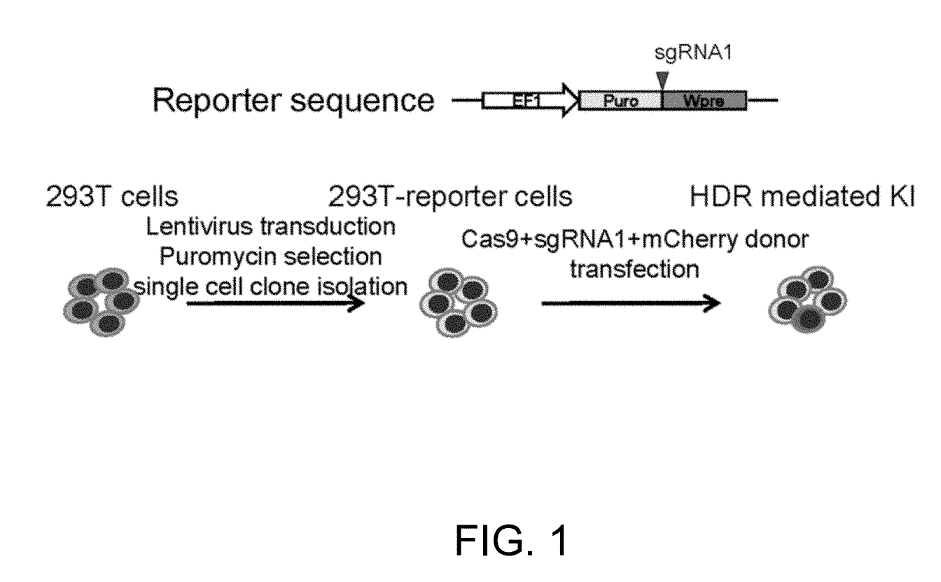

FIG. 1 depicts a scheme of mCherry HDR reporter system. In this experiment, the most commonly used 293 T cells are used to compare the two donor plasmid designs and examine the effects of homology arm (HA) length on HDR efficiency. To this purpose, a reporter system in 293 T cells is established (FIG. 1).

Lentiviral vector Lenti-EF1-Puro-sgRNA1-Wpre containing a sgRNA1 recognition sequence between Puro and Wpre element was constructed in the following steps. The complementary DNA (cDNA) for a puromycin resistant gene (Puro) was amplified by PCR and purified using KAPA HiFi polymerase (KAPA Biosystems) and a GeneJET Gel Extraction Kit (Thermo Fisher Scientific), respectively. The open reading frame of the Puro gene was inserted into a lentiviral vector with the EF1 promoter, used to drive the expression of puromycin resistance gene. Wpre is the woodchuck hepatitis virus posttranscriptional regulatory element. Multiple gene inserts were cloned into lentiviral vector backbones using the NEBuilder HiFi DNA Assembly Kit (New England Biolabs), following manufacturer's instructions. All constructs were verified by Sanger sequencing (MCLAB). Correct clones were grown in CircleGrow Media (MP Biomedicals) and DNA plasmids were purified using Endo-Free Plasmid Maxi Kits (Qiagen). The lentiviral vectors were concentrated a 100-fold by centrifugation at 6000 g for 24 h at 4° C. to reach titers of 2-10×10⁷/mL.

Human embryonic kidney (HEK) 293 T cells were transduced with lentiviral vectors (Lenti EF1-Puro-sgRNA1-Wpre) at a low MOI of 0.1-0.2, and stably transduced cells were selected for by supplementing culture medium with 1 μg/mL puromycin. After one week of antibiotic selection, 293 T reporter lines expressing puromycin resistance and no GFP production were established.

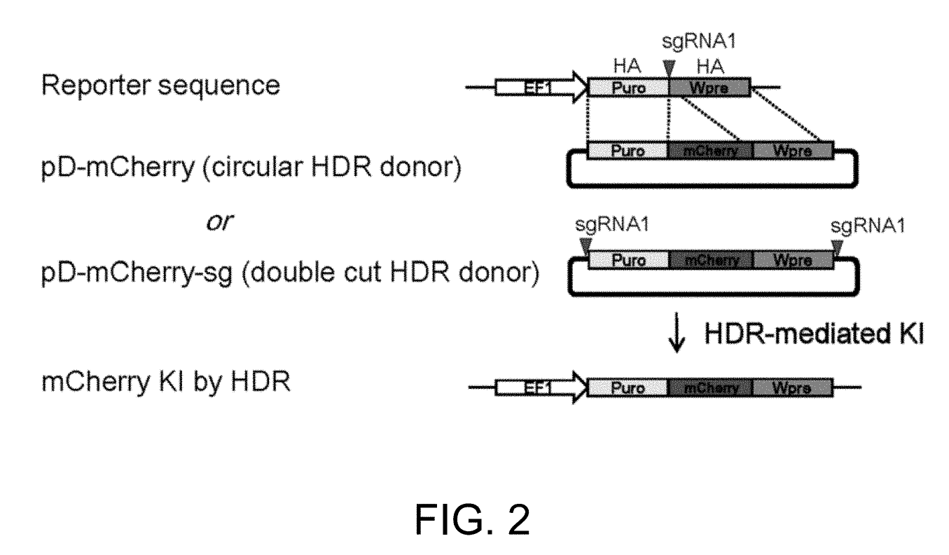

FIG. 2 depicts a schematic design of pD-mCherry donor and pD-mCherry-sg donor. The following plasmids were constructed: pD-mCherry-HA600-600 bp, pD-mCherry-sg-HA600-600 bp, Cas9 plasmid, and sgRNA plasmid.

pD-mCherry-HA600-600 bp is a conventional circular HDR donor and pD-mCherry-sg-HA600-600 bp is a double cut HDR donor in which the Puro-mCherry-Wpre cassette is flanked by two sgRNA1 recognition sequences (FIG. 2). In this experiment, “sg” is tagged in the donor plasmid name to distinguish it from the commonly circular donor. In the two template plasmids, Puro (663 bp) and Wpre (592 bp) are identical and serve as left and right HA, respectively. To simplify naming scheme, the length of Puro and Wpre are unified as 600 bp and the tag HA600-600 bp indicates their HA length. The triangle in FIG. 2 indicates a sgRNA1-PAM sequence that will guide Cas9 to create DSB.

The donor plasmids (pD-mCherry-HA600-600 bp and pD-mCherry-sg-HA600-600 bp) were generated with a CloneJET PCR Cloning Kit (Thermo Scientific). To construct pJET donor plasmids, the homology repair templates were amplified by PCR using KAPA HiFi polymerase (KAPA Biosystems) and purified using a GeneJET Gel Extraction Kit. To clone donor plasmids harboring sgRNA recognition sites (i.e. pD-mCherry-sg-HA600-600 bp), the sgRNA target sequence together with a PAM (NGG) was included in both the forward and the reverse primers. A ligation reaction (20 uL) was performed, on ice, according to the manufacturer's instructions, containing 2× Reaction Buffer (10 uL), pJET1.2/blunt Cloning Vector (50 ng/μL) (1 uL), T4 DNA Ligase (1 uL), purified PCR product (0.15 pmol), and nuclease-free water (remaining volume). The ligation reaction was then briefly vortexed and centrifuged prior to incubation at room temperature (22° C.) for 5-30 min. NEB 5-alpha Competent E. coli cells were then transformed with the ligation product and plated on ampicillin-treated agar plates. Multiple colonies were chosen for Sanger sequencing (MCLAB) to identify the correct clones using the primers pJET1.2-F: CGACTCACTATAGGGAGAGCGGC (SEQ ID NO: 1) and pJET1.2-R: AAGAACATCGATTTTCCATGGCAG (SEQ ID NO: 2).

All Cas9 and sgRNA plasmids were constructed with a NEBuilder HiFi DNA Assembly Kit (New England Biolabs). First, PCR products were produced using KAPA HiFi polymerase (KAPA Biosystems) and purified using a GeneJET Gel Extraction Kit. The linear PCR products were then assembled into plasmids in a DNA assembly reaction (20 uL), on ice, according to the manufacturer's instructions. The reaction contained NEBuilder HiFi DNA Assembly Master Mix (10 uL), equal ratios of PCR products (0.2-0.5 pmols), and deionized water. The ligation reaction was briefly vortexed and centrifuged prior to incubation at 50° C. for 5-30 min. NEB 5-alpha Competent E. coli cells were then transformed with the assembled DNA products and plated on ampicillintreated agar plates. Multiple colonies were chosen for Sanger sequencing (MCLAB) to identify the correct clones using the primer U6-F: GGGCAGGAAGAGGGCCTAT (SEQ ID NO: 3). The sgRNA1 sequence was GGTGCAGATGAACTTCA (SEQ ID NO: 4).

Following co-transfection with a promoterless mCherry donor plasmid (pD-mCherry-HA600-600 bp or pD-mCherry-sg-HA600-600 bp) and two plasmids encoding Cas9 and sgRNA1, mCherry is knocked into the target locus by HDR and the cells become mCherry⁺ (FIG. 2). For transfection of HEK 293 T cells, Lipofectamine 3000 (Life Technologies) was used according to manufacturer's instructions. After co-transfection with promoterless mCherry donor (pD-mCherry-HA600-600 bp or pD-mCherry-sg-HA600-600 bp) and two plasmids encoding Cas9 and sgRNA1, the 293 T reporter cells use the donor to repair DSB by HDR pathway leading to the integration and expression of mCherry. Although NHEJ insertion of donor may occur in this system, these cells would remain mCherry⁻.

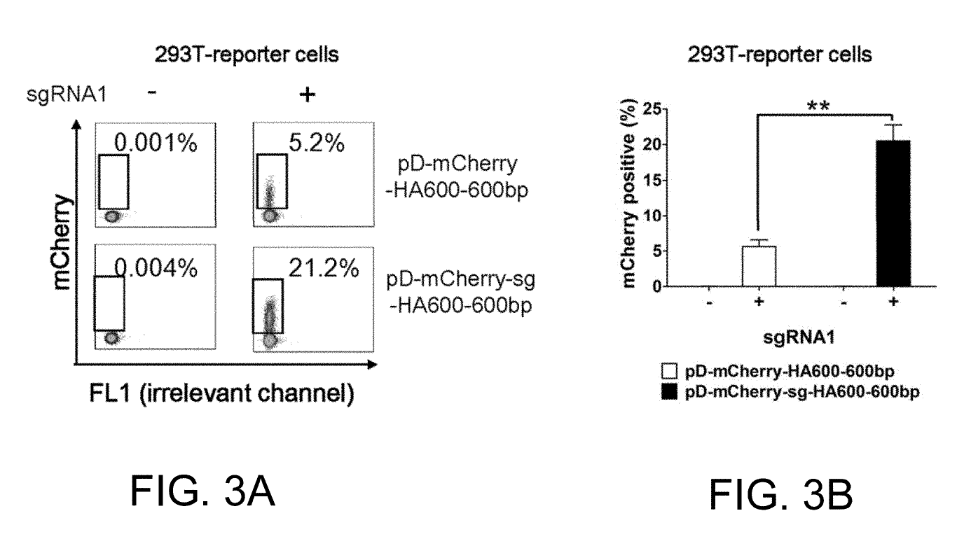

FIG. 3A and FIG. 3B depict flow cytometry analysis of 293 T reporter cell after co-transfection of Cas9/conventional pD-mCherry donor, compared to Cas9/pD-mCherry-sg donor. One week after co-transfection, the portion of mCherry⁺ cells detected by flow cytometry (FACS) reflects HDR efficiency. Flow cytometry analysis indicated that the efficiency of Cas9/conventional pD-mCherry mediated knockin of mCherry was only around 5%. In contrast, the efficiency of Cas9/pD-mCherry-sg mediated knockin of mCherry was around 20%. That is, a fourfold increase in the portion of mCherry⁺ cells with double cut donor pD-mCherry-sg-HA600-600 bp compared to pDmCherry-HA600-600 bp was observed (FIG. 3A, 3B). Based on the above, in vivo cleavable donor plasmid can increase HDR; this can be achieved by sandwiching the donor vector with two sgRNA recognition sequences. When the Cas9/sgRNA complex surveys the genome and plasmids, it creates genomic DSB and linearizes donor plasmids simultaneously, thus synchronizing the demand and supply of homologous sequences and thereby increasing HDR. Moreover, in commonly known method for cleaving gDNA and donor plasmid, two sgRNAs were used, one for creating genomic DSB and another for releasing donor template from the plasmid. This design increases complexity and occasionally may not be able to perfectly synchronize the demand and supply of homologous sequences, because the cleavage efficiencies of the two distinct sgRNAs may not be identical. In contrast, in present embodiment, one sgRNA is used to target both gDNA and the donor plasmid.

### Example 2: High HDR Efficiency is Achieved in 293 T Cells by Double Cut HDR Donors Even with Short Homology Arm

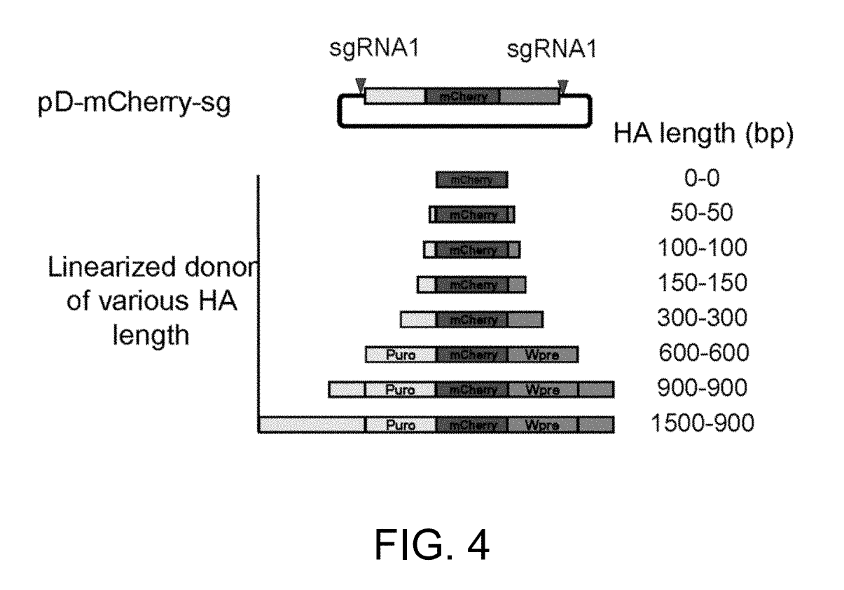

FIG. 4 depicts a schematic design of pD-mCherry-sg donor with HA in the range of 0˜1500 bp in length. In this experiment, a series of donors with HA in the range of 50-1500 bp in length are designed (50 bp, 100 bp, 150 bp, 300 bp, 600 bp, 900 bp, and 1500 bp). All of the double cut donors contain target sequence of sgRNA1 to flank the donor plasmids and can be linearized inside cells after co-transfection with Cas9 and sgRNA1 (FIG. 4). As a control, a series of conventional circular HDR donors with various HA in the range of 300-1500 bp are also designed (300 bp, 600 bp, 900 bp, and 1500 bp). All of the donor plasmids used in this experiment were generated with a CloneJET PCR Cloning Kit (Thermo Scientific).

Following co-transfection with a promoterless mCherry donor plasmid (pD-mCherry donor or pD-mCherry-sg donor) and two plasmids encoding Cas9 and sgRNA1 using Lipofectamine system as described above.

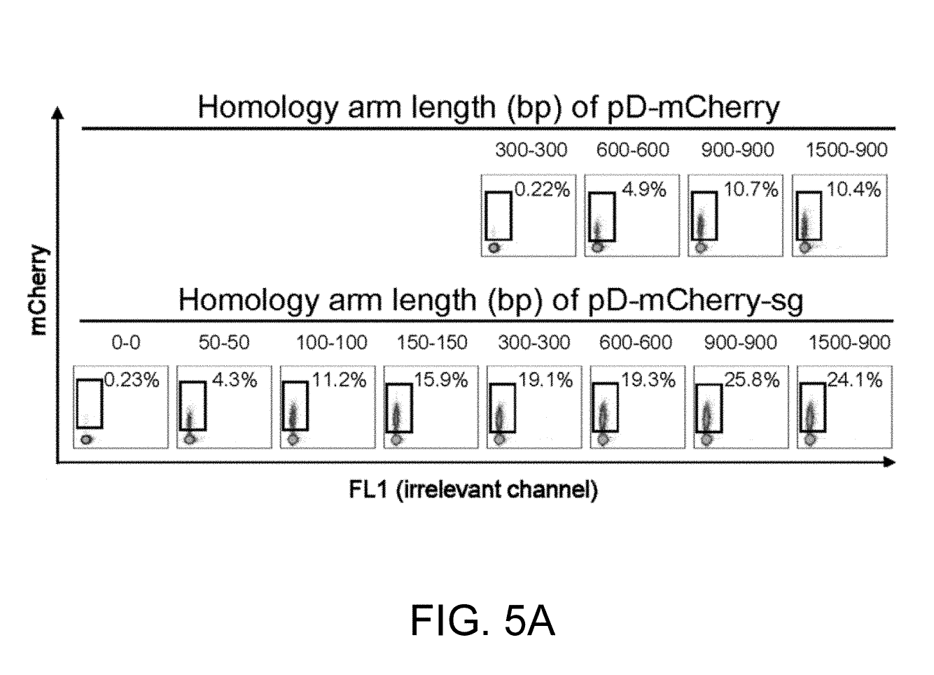

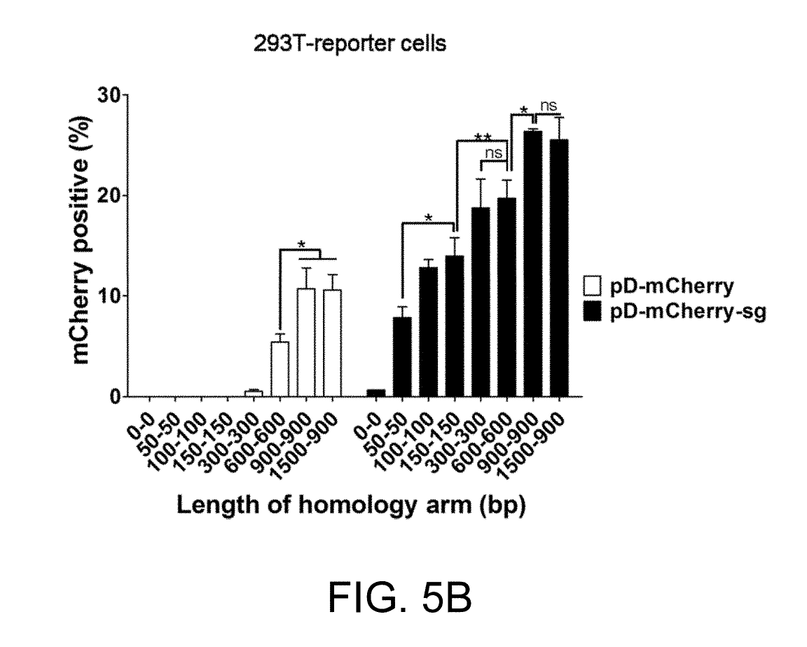

FIG. 5A and FIG. 5B depict flow cytometry analysis of 293 T reporter cell after co-transfection of Cas9/conventional pD-mCherry donor, compared to Cas9/pD-mCherry-sg donor. One week after co-transfection, the portion of mCherry⁺ cells detected by flow cytometry (FACS) reflects HDR efficiency. When HA of the conventional circular donors (pD-mCherry donor) increased from 300 bp through 600˜900 bp, HDR efficiency increased to 10% (FIG. 5A, 5B). In this experiment, circular donors with shorter HA did not construct because HDR efficiency was as low as 0.22% when HA is 300 bp (FIG. 5A, 5B).

To illustrate here, double cut donors may increase the events of NHEJ, thus the donor with 0 bp HA (pD-mCherry-sg-HAO-O bp) was served as a control of events of NHEJ. When 293 T cells were transfected with pD-mCherry-sg-HAO-O bp, only 0.6% of cells expressed mCherry (mCherry⁺), suggesting that NHEJ contributes only minimally to the percentage of mCherry⁺ cells (FIG. 5A, 5B). This result validates the use of percentage of mCherry⁺ cells as an indicator of HDR efficiency. Flow cytometry analysis indicated that the HA as short as 50 bp led to a 6-10% HDR efficiency. With the increase of HA from 50 bp through 100-150 bp, a twofold increase in HDR efficiency was observed. A further increase of HA in double cut donors led to a gradual increase of HDR efficiency to 26% (FIG. 5A, 5B).

Taken together, the above results conducted in 293 T cells suggest that a short HA of 300 bp in circular donor is inefficient for HDR, whereas the same HA in double cut donor leads to significant HDR. The double cut donor system not only increases the HDR efficiency, but also reduces the demand for HA length.

### Example 3: Enhanced HDR Editing and Suppressed NHEJ Editing at the CTNNB1 Locus in iPSCs with Double Cut HDR Donors

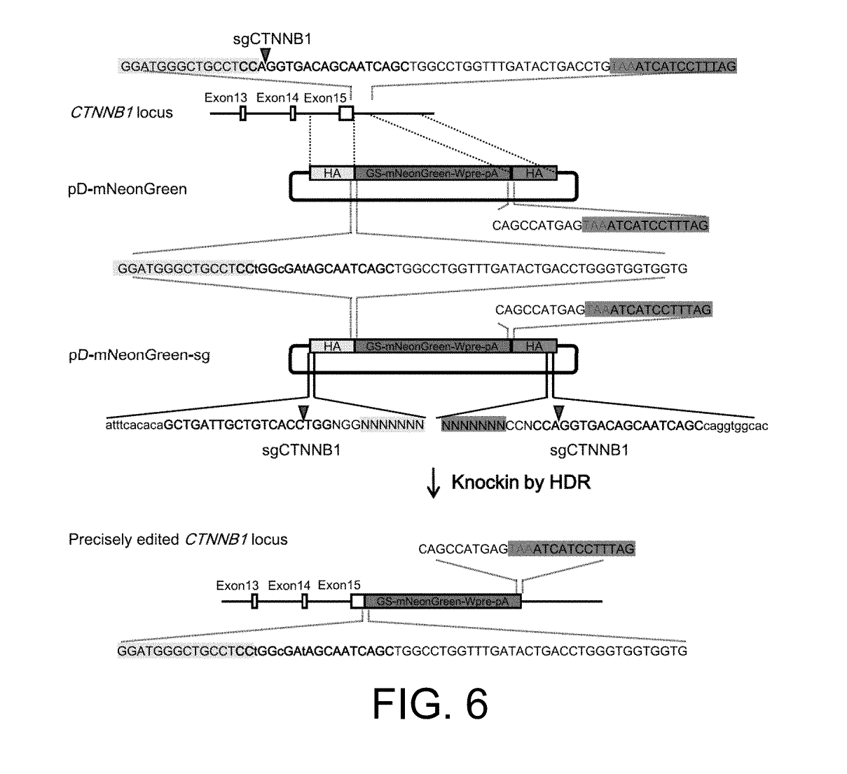

FIG. 6 depicts a scheme of genome editing at the CTNNB1 locus in iPSCs.

In this experiment, a human iPSC line was used due to its significance in regenerative medicine and well-known difficulty in comparison to 293 T cells. A targeting scheme is shown in FIG. 6 for expressing a mNeonGree protein after knockin of mNeonGree sequence into the endogenous CTNNB1 locus. CTNNB1 is a pivotal gene in the canonical WNT pathway that is constitutively expressed in iPSCs and other cells. A sgCTNNB1 is used to target 39 bp before the stop codon (FIG. 6), which showed a 60% cleavage efficiency in iPSCs (data not shown). The sgCTNNB1 sequence was GCTGATTGCTGTCACCTGG (SEQ ID NO: 5).

In this experiment, a series of donors with GS-mNeonGreen-Wpre-polyA sequence being flanked by HA to this locus on both sides with various lengths were constructed. Silent mutations inside the gene were introduced to prevent cleavage in the middle of the donor by sgCTNNB1. GS is a quadruple GGGGS linker and mNeonGreen is a bright fluorescent protein. Similar to the above design, a series of conventional circular donors (pD-mNeonGreen) with HA in the range of 150˜2000 bp (150 bp, 300 bp, 600 bp, 1000 bp, 1500 bp and 2000 bp) and double cut donors (pD-mNeonGreen-sg) with HA of 50˜2000 bp (50 bp, 100 bp, 150 bp, 300 bp, 600 bp, 1000 bp, 1500 bp and 2000 bp) were constructed.

In this experiment, all of the donor plasmids used in this experiment were generated with a CloneJET PCR Cloning Kit (Thea no Scientific). To construct donor plasmid targeting CTNNB1 at 39 bp before the stop codon, the left and right HA of the donor plasmid were amplified from human genomic DNA; with the stop codon being removed and in-frame linked with the GS sequence (a quadruple of GGGGS peptides); the insert mNeonGreen-Wpre-polyA was amplified from another vector. For the double cut donors (pD-mNeonGreen-sg), a sgCTNNB1 target sequence together with the PAM sequence (GCTGATTGCTGTCACCTGGAGG) (SEQ ID NO: 6) was tagged at a location outside of the two HA.

Following co-transfection with a donor plasmid (pD-mNeonGreen or pD-mNeonGreen-sg) and two plasmids encoding Cas9 and sgRNA1 into the human iPSCs. For transfection of human iPSCs, cells were electroporated using the Lonza Nucleofector system (Lonza). After co-transfection, HDR-mediated knockin leads to the formation of a CTNNB1-mNeonGreen fusion protein that fluoresces green.

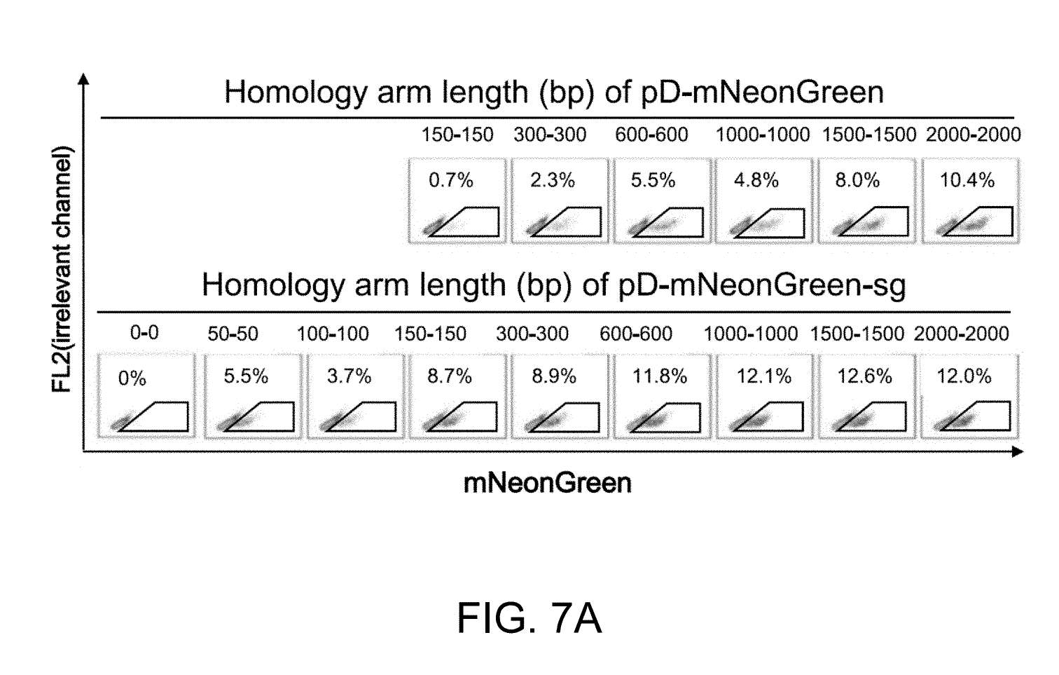

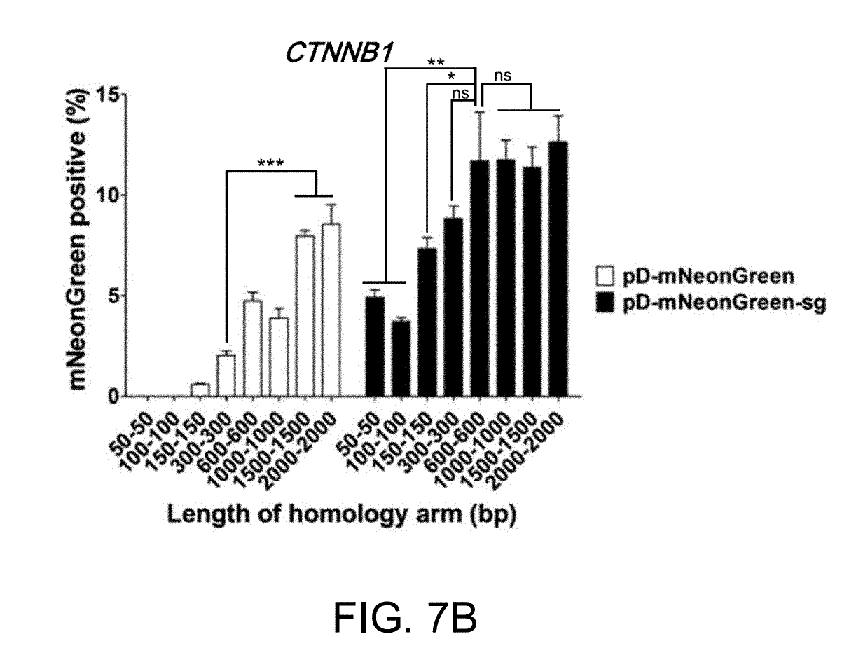

FIG. 7A and FIG. 7B depict flow cytometry analysis of human iPSCs after co-transfection of Cas9/conventional pD-mNeonGreen donor, compared to pD-mNeonGreen-sg donor. As the HA length of pD-mNeonGreen donors increased from 150 bp to 2000 bp, HDR efficiency at CTNNB1 progressively increased from 0.7% to 11% (FIG. 7A, 7B). In comparison, pD-mNeonGreen-sg donors showed 4-5% HDR efficiency even with short HA of 50˜100 bp. An increase of HA from 150 bp through 300-600 bp led a gradual increase of HDR from 8% to 12%. Further elongation of HA to 1000 bp, 1500 bp, or 2000 bp in the double cut donors showed 12% HDR efficiency (FIG. 7A, 7B). Consistent with the notion that homologous recombination depends on HA, a donor with 0 bp HA (pD-mCherry-sg-HAO-0 bp) showed 0% HDR efficiency (FIG. 7A).

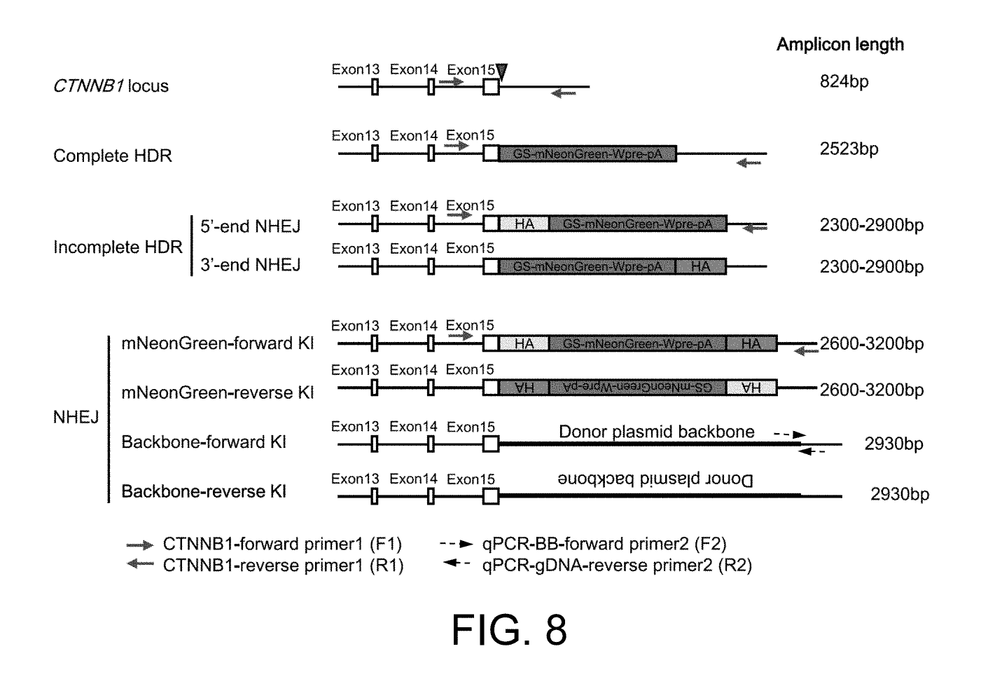

FIG. 8 depicts a scheme of different knockin patterns. Besides precise editing by HDR, there are two major possibilities of partial HDR and four possibilities of NHEJ insertions (FIG. 8). To investigate these events in bulk iPSCs, CTNNB1 forward primer-F1 (GTGGCCTGGCACTGAGTAAT) (SEQ ID NO: 7) and CTNNB1 reverse primer-R1 (CTCAGCAACTCTACAGGCCA) (SEQ ID NO: 8) were designed to specifically amplify the genomic locus without amplifying donors with HA of 50 bp, 100 bp, 150 bp, or 300 bp. The expected amplicon length is 824 bp for wild-type allele and 2000˜4000 bp for the edited allele (FIG. 8).

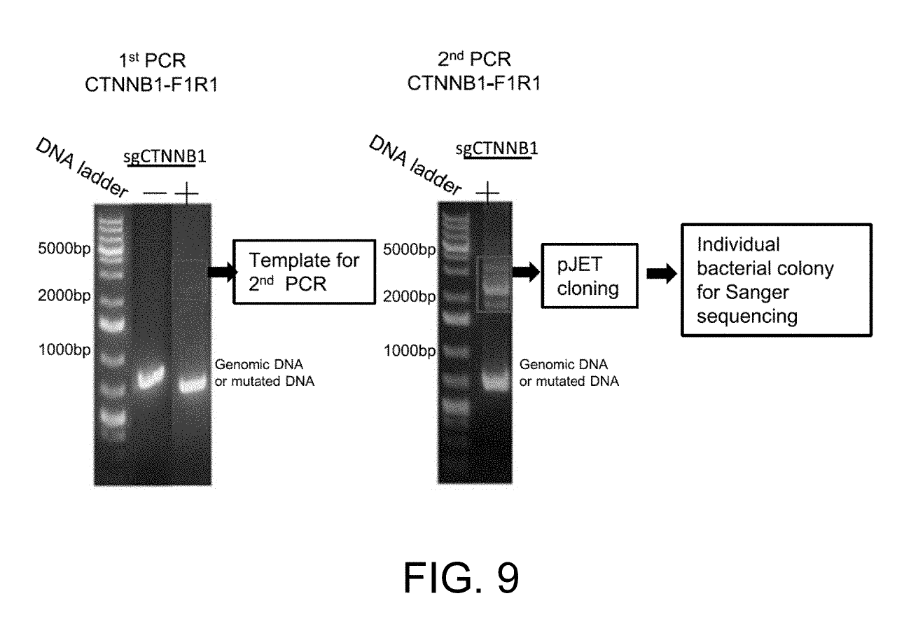

Human iPSCs were harvested at day 3 after cotransfection of Cas9/sgRNA and donors (pD-mNeonGreen-sg-HA50-50 bp, pD-mNeonGreen-sg-HA100-100 bp, pD-mNeonGreen-sg-HA150-150 bp, or pD-mNeonGreen-sg-HA300-300 bp) for DNA extraction. The CTNNB1 target sequences was amplified with KAPA HiFi DNA polymerase by PCR twice. For the first-round PCR at CTNNB1 locus, CTNNB1 forward primer-F1 and CTNNB1 reverse primer-R1 were used with the PCR cycling condition being 98° C. for 5 min, followed by 98° C. for 5 s, 68° C. for 1 min for 30 cycles. FIG. 9 shows a result of PCR analysis for knockin pattern. The bands in the size range of 2-4 kb in the first-round PCR were cut out and purified using the GeneJET Gel Extraction Kit. For second-round PCR, 1 ng of purified primary PCR products was amplified using the same pair of primers and cycling conditions. The bands in the range of 2-4 kb from the second PCR were purified and cloned into the pJET vector (FIG. 9). Approximately 50 individual bacterial colonies from each condition were picked for Sanger sequencing. Clones with high-quality sequencing data at both ends were aligned with expected HDR knockin sequence and donor plasmid sequence by BLAST.

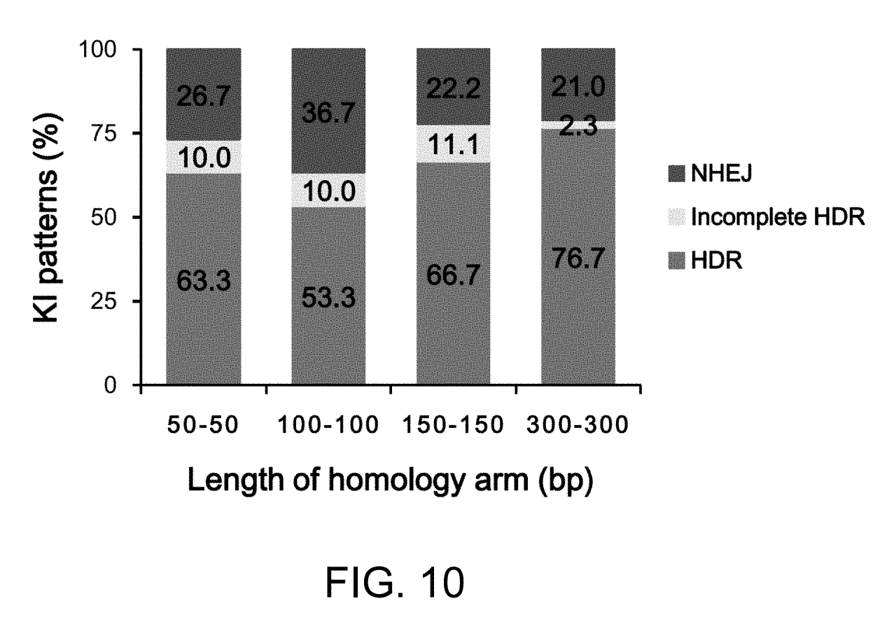

Table 1 shows a summary of Sanger sequencing results. FIG. 10 shows a distribution of different knockin patterns by double cut HDR donors with different HA lengths. The majority of knockin events were HDR, with a about 77% precise insertion rate being observed with HA of 300 bp (Table 1, FIG. 10).

TABLE 1

| | Number of clones | | | | | | | | | |
|---|---|---|---|---|---|---|---|---|---|---|
| | | | | | NHEJ | | | | | |
| | Complete | | Incomplete | | mediated | | | | | |
| HA | HDR | | HDR | | KI | | | | | |
| length | No | with | 5-end | 3-end | mNeonGreen- | mNeonGreen- | Backbone- | Backbone- | Total | HDR |
| (bp) | mutation | mutation | NHEJ | NHEJ | forward | reverse | forward | reverse | number | (%) |
| 50-50 | 19 | 0 | 0 | 3 | 0 | 1 | 3 | 4 | 30 | 63.3 |
| 100-100 | 16 | 0 | 1 | 2 | 1 | 0 | 9 | 1 | 30 | 53.3 |
| 150-150 | 24 | 0 | 1 | 3 | 1 | 0 | 7 | 0 | 36 | 66.7 |
| 300-300 | 33 | 0 | 0 | 1 | 0 | 0 | 5 | 4 | 43 | 76.7 |

To quantitate the occurrence of NHEJ-mediated insertion of large pieces from donor plasmid, the quantitative PCR (qPCR) were conducted. Multiple forward backbone insertion was detected (Table 1), which can be used as a surrogate indicator to assess NHEJ. qPCR-BB-forward primer2 (F2, CACTCATTAGGCACCCCAGG) (SEQ ID NO: 9) and qPCR-gDNA-reverse primer2 (R2, CCCACCCTACCAACCAAGTC) (SEQ ID NO: 10) were designed to specifically amplify this particular NHEJ event (FIG. 8).

Human iPSCs were harvested at day 3 after cotransfection of Cas9/sgRNA and donors (pD-mNeonGreen-sg-HA50-50 bp, pD-mNeonGreen-sg-HA100-100 bp, pD-mNeonGreen-sg-HA150-150 bp, pD-mNeonGreen-sg-HA300-300 bp, pD-mNeonGreen-sg-HA600-600 bp, pD-mNeonGreen-sg-HA1000-1000 bp, pD-mNeonGreen-sg-HA1500-1500 bp, or pD-mNeonGreen-sg-HA2000-2000 bp) for DNA extraction. The real-time PCR reaction system (20 μL) consisted of 10 μL of SYBR Green qPCR Master Mix (2×), 1 μL each of F2 (qPCR-BB-forward primer2) and R2 (qPCR-gDNA-reverse primer2), and 100 ng gDNA from a cell sample from day 3. The cycling conditions were 95° C. for 3 min, followed by 95° C. for 15 s, 64° C. for 20 s, and 72° C. for 30 s, for 35 cycles. The GAPDH gene, a housekeeping gene, was used to normalize the qPCR data.

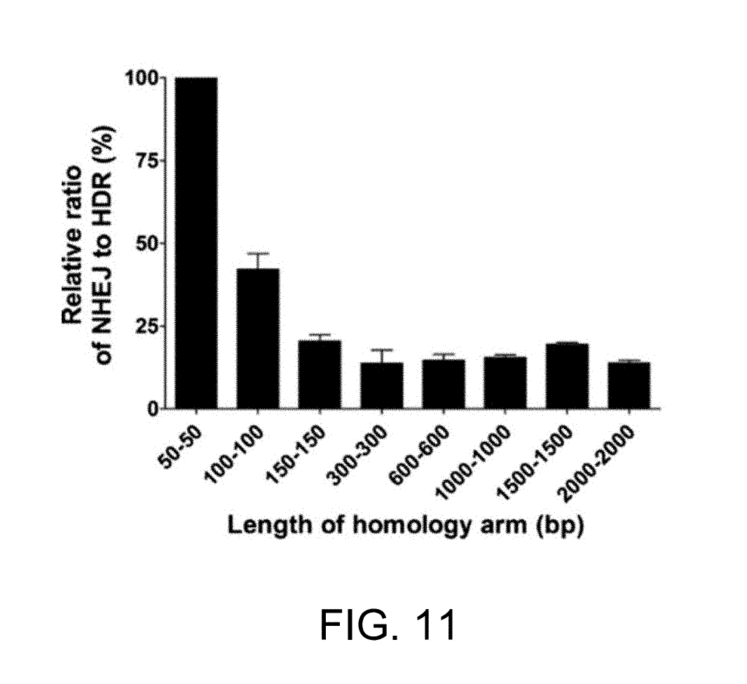

FIG. 11 shows a quantitative PCR (qPCR) analysis of donor plasmid backbone-forward insertion. The relative ratio of NHEJ/HDR was calculated by qPCR data (which is designed to amplify NHEJ insertion of plasmid backbone) divided by percentage of mNeonGreen⁺ cells (which reflects HDR insertion) in a certain sample. With the increase of HA from 50 bp to 300 bp, the relative NHEJ was significantly dropped by 80%, whereas further increase of the HA length did not lead to a further decrease in NHEJ (FIG. 3 h), suggesting that a 300 bp homology on both arms of double cut donors is sufficient to increase HDR and/or suppress NHEJ.

### Example 4: Reducing the Length of Replaced Sequence Surrounding DSB Site Enhances HDR

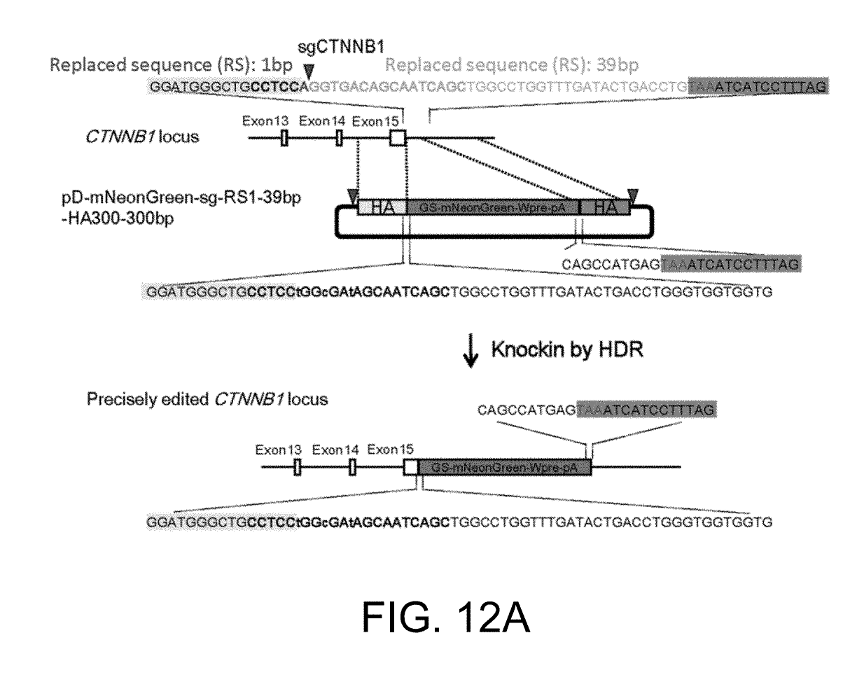

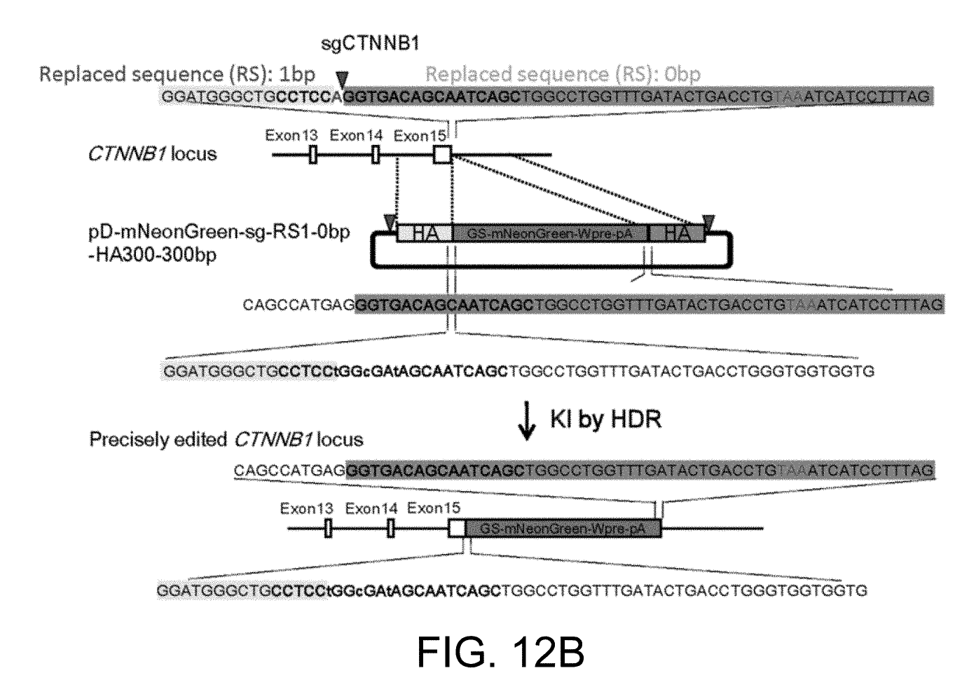

FIG. 12A depicts a schematic illustration of the replaced sequence (RS) in pD-mNEonGreen-sg-RS1-39 bp-HA300-300 bp donor. FIG. 12B depicts a schematic illustration of the replaced sequence (RS) in pD-mNEonGreen-sg-RS1-0 bp-HA300-300 bp.

In this experiment, new pDmNeonGreen-sg of 300 bp homology was constructed. The pD-mNEonGreen-sg-RS1-39 bp-HA300-300 bp donor was constructed using a similar method to former pD-mNeonGreen-sg-RS1-39 bp-HA300-300 bp (same as the donor pD-mNeonGreen-HA300-300 bp) in example 3, and the difference thereof is that the right HA in the new donor pD-mNeonGreen-sg-RS1-0 bp-HA300-300 bp extends to the cut site on genomic DNA, making the replaced sequence (RS) to be 0 bp on the right side (FIG. 12A and FIG. 12B).

Following co-transfection with a donor plasmid (pD-mNEonGreen-sg-RS1-0 bp-HA300-300 bp or pD-mNeonGreen-sg-RS1-39 bp-HA300-300 bp) and two plasmids encoding Cas9 and sgRNA1 into the human iPSCs. For transfection of human iPSCs, cells were electroporated using the Lonza Nucleofector system (Lonza). After co-transfection, HDR-mediated knockin leads to the formation of a CTNNB1-mNeonGreen fusion protein that fluoresces green.

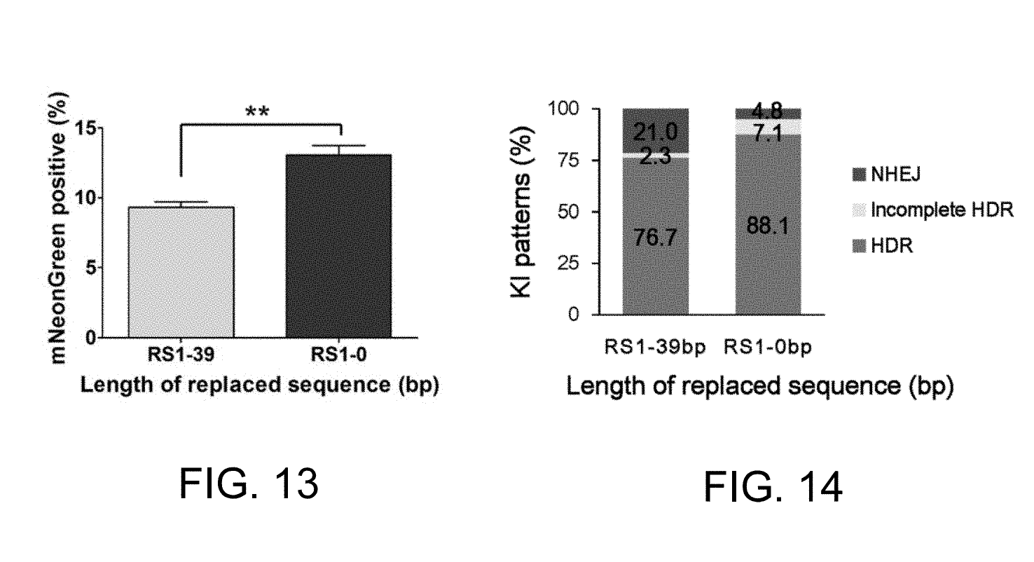

FIG. 13 depict flow cytometry analysis of human iPSCs after co-transfection of Cas9/pD-mNEonGreen-sg-RS1-0 bp-HA300-300 bp donor, compared to pD-mNeonGreen-sg-RS1-39 bp-HA300-300 bp donor. The decrease of RS from 1-39 bp to 1-0 bp led to a 45% improvement in HDR efficiency.

In this experiment, the NHEJ insertion events in genome editing system were also determined. The method of determining NHEJ-mediated and HDR-mediated knockin have been described above (see example 3). Table 2 shows a summary of Sanger sequencing results. FIG. 14 shows a distribution of different knockin patterns when using two donors. The HDR occurrence in bulk population increased from 77% to 88% (FIG. 14, Table 2). Knockin pattern analysis showed that the proportion of NHEJ decreased from 21% to 5% (FIG. 14).

TABLE 2

| | Number of clones | | | | | | | | | |
|---|---|---|---|---|---|---|---|---|---|---|
| | | | | | NHEJ | | | | | |
| | Complete | | Incomplete | | mediated | | | | | |
| | HDR | | HDR | | KI | | | | | |
| | No | with | 5-end | 3-end | mNeonGreen- | mNeonGreen- | Backbone- | Backbone- | Total | HDR |
| | mutation | mutation | NHEJ | NHEJ | forward | reverse | forward | reverse | number | (%) |
| RS1-39 bp | 33 | 0 | 0 | 1 | 0 | 0 | 5 | 4 | 43 | 76.7 |
| RS1-0 bp | 37 | 0 | 3 | 0 | 0 | 0 | 0 | 2 | 42 | 88.1 |

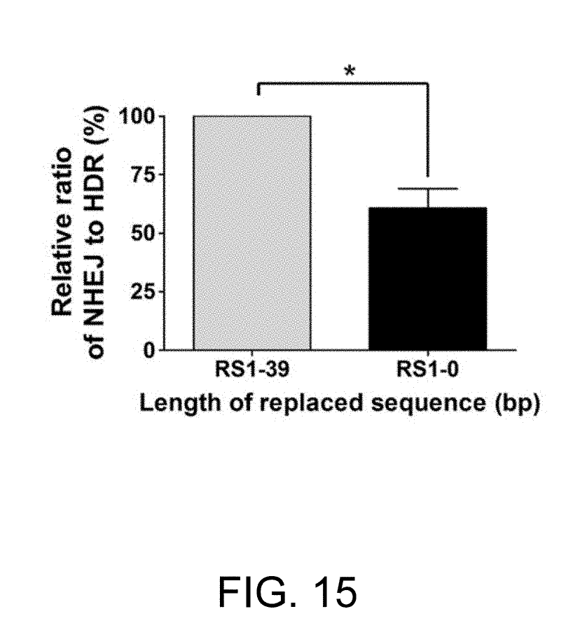

To quantitate the occurrence of NHEJ-mediated insertion of large pieces from donor plasmid, the quantitative PCR (qPCR) were conducted. FIG. 15 shows a quantitative PCR (qPCR) analysis of donor plasmid backbone-forward insertion. In agreement with this result, qPCR that examines backbone insertion indicated a ˜40% decrease in the NHEJ/HDR ratio.

In addition, 293 T reporter lines engineered with either a 50 bp or 200 bp sequence that need to be replaced on one or two arms before HDR were used. HDR rate was significantly decreased with replaced sequence of 200 bp in one arm. When replaced sequence was present on both arms, an up to 50% decrease in HDR was observed (data not shown). Taken together, these results indicate that in order to achieve high-level HDR and minimize NHEJ, two HA of the double cut donors should be identical to the sequences surrounding DSB. Minimizing the replaced sequence surrounding the DSB increases HDR and suppresses NHEJ-mediated insertion.

### Example 5: High HDR Efficiency and Low NHEJ Occurrence at the PRDM14 Locus by Double Cut HDR Donor with Short Homology Arms

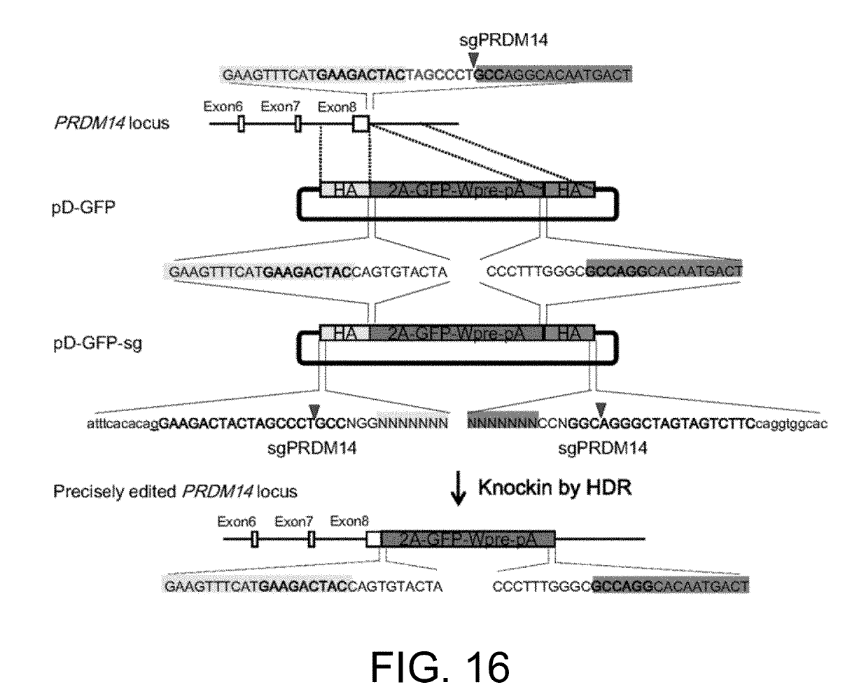

FIG. 16 depicts a scheme of genome editing at the PRDM14 locus in iPSCs. PRDM14 is a regulator of pluripotency. A targeting scheme is shown in FIG. 16 for expressing a GFP protein after knockin of GFP sequence into the endogenous PRDM14 locus. An sgPRDM14 was designed to target the sequence surrounding the stop codon, with cleavage site at 4 bp downstream of the stop codon (FIG. 16), and the cleavage efficiency of this sgPRDM14 was ˜30% in iPSCs (data not shown). The sg PRDM14 sequence was GAAGACTACTAGCCCTGCC (SEQ ID NO: 11).

In this experiment, a series of donors with 2A-GFP-Wpre-polyA sequence being flanked by HA to this locus on both sides with various lengths were constructed, in which 2A sequence is a ribosome-skipping sequence allowing for co-translation of PRDM14 and GFP. Similar to the above design, a series of conventional circular donors (pD-GFP) with HA in the range of 150-2000 bp (150 bp, 300 bp, 600 bp, 1000 bp, 1500 bp and 2000 bp) and double cut donors (pD-GFP-sg) with HA of 50˜2000 bp (50 bp, 100 bp, 150 bp, 300 bp, 600 bp, 1000 bp, 1500 bp and 2000 bp) were constructed.

In this experiment, all of the donor plasmids used in this experiment were generated with a CloneJET PCR Cloning Kit (Thermo Scientific). To construct donor plasmids targeting the PRDM14 stop codon, the left and right HA were amplified from human genomic DNA, with the stop codon being removed and in-frame linked with the 2A sequence; the insert 2A-GFP-Wpre-polyA was amplified from another vector. For the double cut donors (pD-GFP-sg), a sgPRDM14 target sequence together with the PAM sequence (GGAAGACTACTAGCCCTGCCAGG) (SEQ ID NO: 12) was tagged to the regions flanking the upstream and downstream HA.

Following co-transfection with a donor plasmid (pD-GFP or pD-GFP-sg) and two plasmids encoding Cas9 and sgRNA1 into the human iPSCs. For transfection of human iPSCs, cells were electroporated using the Lonza Nucleofector system (Lonza). After co-transfection, HDR-mediated knockin leads to the expression GFP and the cells become GFP⁺ (FIG. 16).

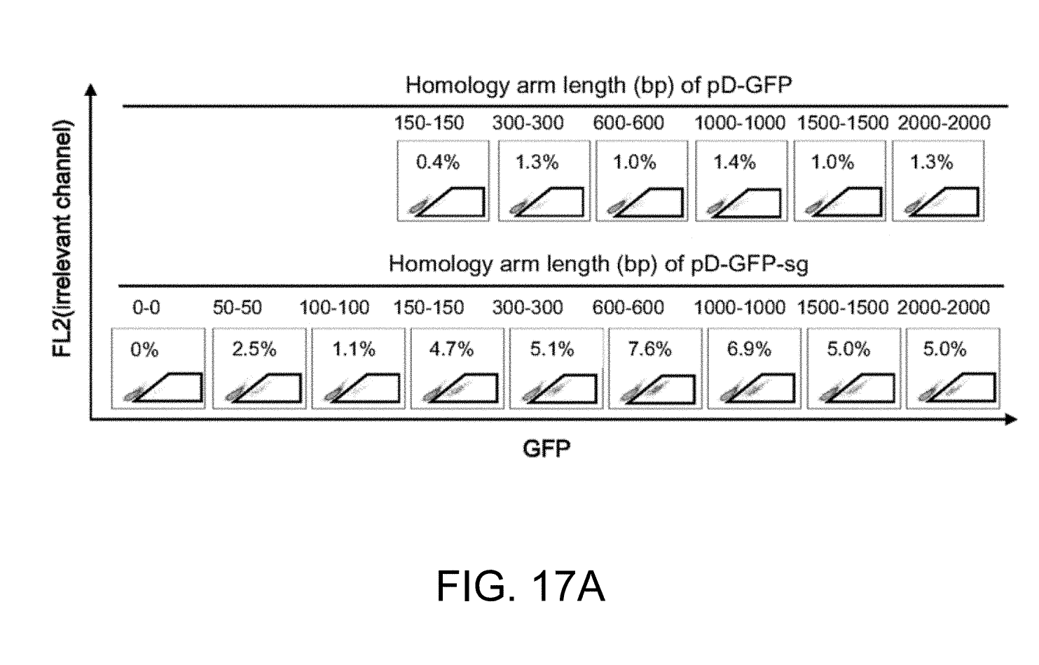

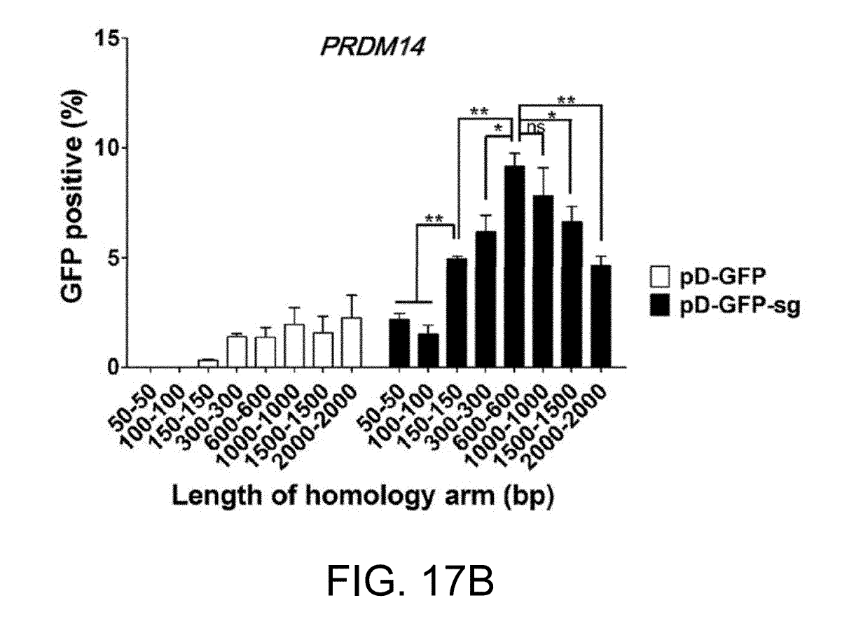

FIG. 17A and FIG. 17B depict flow cytometry analysis of human iPSCs after co-transfection of Cas9/conventional pD-GFP donor, compared to pD-GFP-sg donor. FACS analysis showed that HDR efficiencies have a tendency to increase along with the elongation of HA when using pD-GFP, even though the general HDR efficiency was relatively low (1-3%) (FIG. 17A, 17B). The pD-GFP-sg double cut donors showed a dramatic increase in HDR efficiency when HA length was extended from 50˜100 bp (1-3%) to 600 bp (9%) (FIG. 17A, 17B). A donor with 0 bp HA (pD-GFP-sg-HAO-0 bp) was used to control the events of NHEJ, which showed a ˜0% HDR efficiency (FIG. 17A).

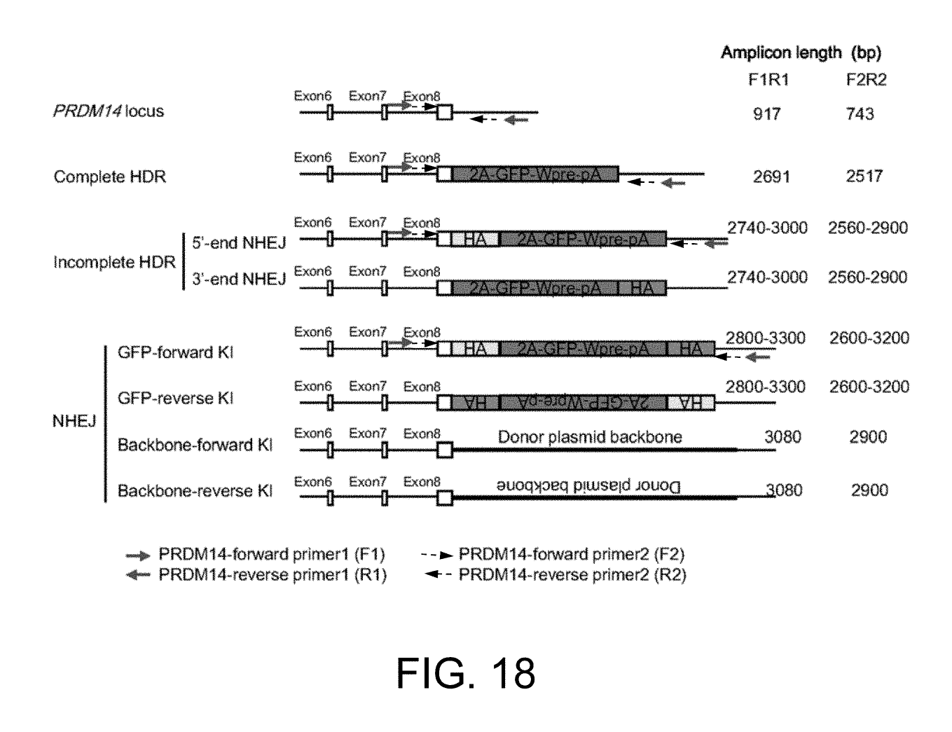

FIG. 18 depicts a scheme of different knockin patterns. To investigate different knockin patterns in bulk iPSCs, PRDM14 forward primer-F1 (CCAGCCTGCAATCTGCTTTT) (SEQ ID NO: 13) and PRDM14 reverse primer-R1 (GCCAACTGCAGGGACTTCTA) (SEQ ID NO: 14) for the first-round PCR and PRDM14 forward primer-F2 (GACCAGGAGTGCTCTATGGC) (SEQ ID NO: 15) and PRDM14 reverse primer-R2 (AGGAAATAGAGAGAATCCGAATCTC) (SEQ ID NO: 16) for the second-round PCR were designed to specifically amplify the genomic locus without amplifying donors with HA of 50 bp, 100 bp, 150 bp, or 300 bp.

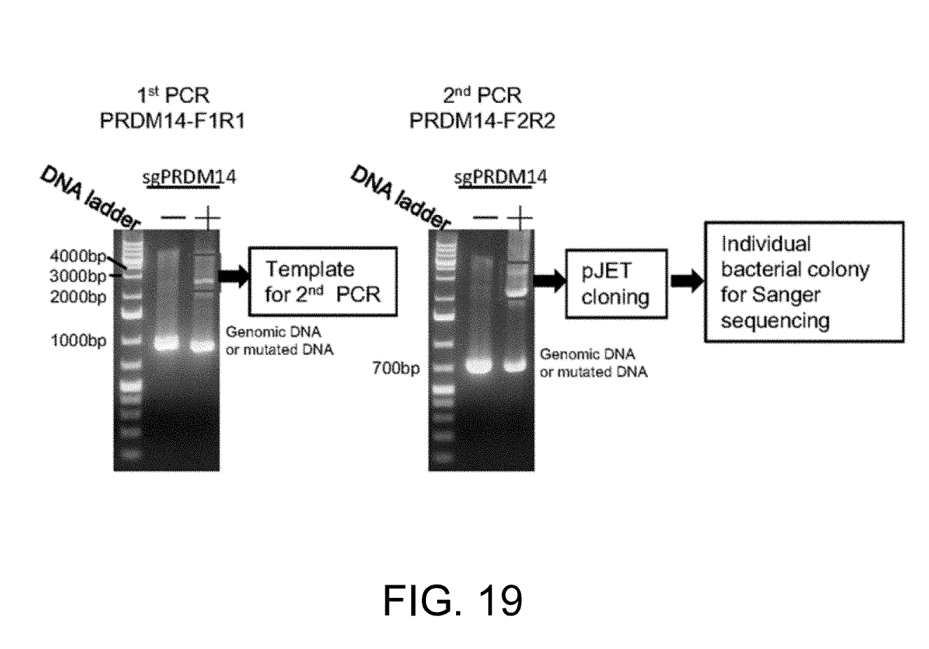

Human iPSCs were harvested at day 3 after cotransfection of Cas9/sgRNA and donors (pD-GFP-sg-HA50-50 bp, pD-GFP-sg-HA100-100 bp, pD-GFP-sg-HA150-150 bp, or pD-GFP-sg-HA300-300 bp) for DNA extraction. The PRDM14 target sequences was amplified with KAPA HiFi DNA polymerase by PCR twice. For the first-round PCR at PRDM14 locus, PRDM14 forward primer-F1 and PRDM14 reverse primer-R1 were used with the PCR cycling condition being 98° C. for 5 min, followed by 98° C. for 5 s, 68° C. for 1 min for 30 cycles. FIG. 19 shows a result of PCR analysis for knockin pattern. The bands in the size range of 2-4 kb in the first-round PCR were cut out and purified using the GeneJET Gel Extraction Kit. For second-round PCR, 1 ng of purified primary PCR products was amplified using PRDM14 forward primer-F1 and PRDM14 reverse primer-R1 and the same cycling conditions. The bands in the range of 2-4 kb from the second PCR were purified and cloned into the pJET vector (FIG. 19). Approximately 40 individual bacterial colonies from each condition were picked for Sanger sequencing. Clones with high-quality sequencing data at both ends were aligned with expected HDR knockin sequence and donor plasmid sequence by BLAST.

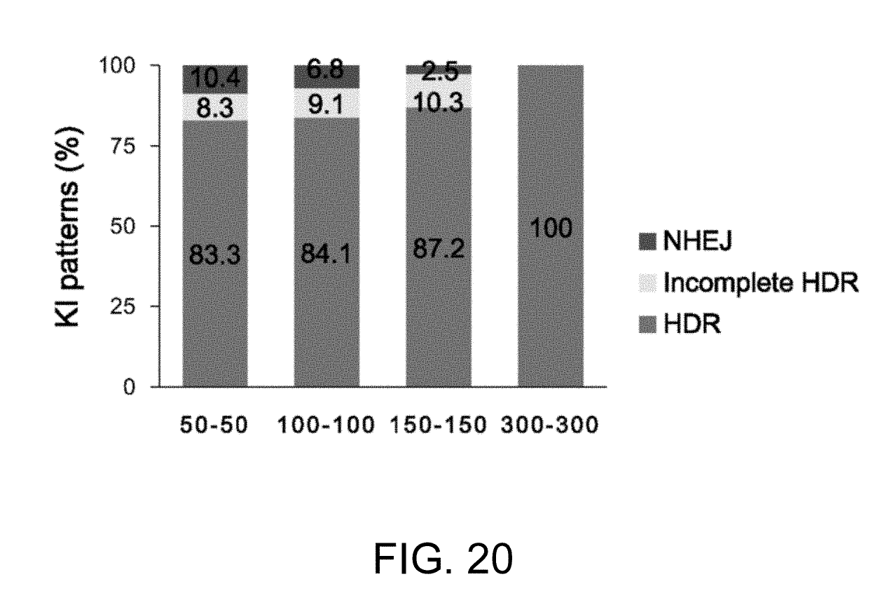

Table 3 shows a summary of Sanger sequencing results. FIG. 20 shows a distribution of different knockin patterns by double cut HDR donors with different HA lengths. As HA length increased, from 50 bp to 300 bp, the HDR rate increased from 80% to 100% (Table 3, FIG. 20). Taken together, these results indicate that high-level HDR can be achieved with double cut donor.

TABLE 3

| | Number of clones | | | | | | | | | |
|---|---|---|---|---|---|---|---|---|---|---|
| | | | | | NHEJ | | | | | |
| | Complete | | Incomplete | | mediated | | | | | |
| HA | HDR | | HDR | | KI | | | | | |
| length | No | with | 5-end | 3-end | mNeonGreen- | mNeonGreen- | Backbone- | Backbone- | Total | HDR |
| (bp) | mutation | mutation | NHEJ | NHEJ | forward | reverse | forward | reverse | number | (%) |
| 50-50 | 38 | 2 | 3 | 1 | 2 | 0 | 0 | 3 | 48 | 79.2 |
| 100-100 | 37 | 0 | 3 | 1 | 0 | 0 | 3 | 0 | 44 | 84.1 |
| 150-150 | 34 | 0 | 2 | 2 | 0 | 0 | 1 | 0 | 39 | 87.2 |
| 300-300 | 45 | 0 | 0 | 0 | 0 | 0 | 0 | 0 | 45 | 100 |

### Example 6: Double Cut Donor-Mediated HDR can be Further Improved by Cell Cycle Regulators

[Effects of Small Molecules on HDR Efficiency at the CTNNB1 or PRDM14 Locus in iPSCs]

In this experiment, the verified highly efficient pD-sg-HA300-300 bp double cut donor and multiple small molecules were used to examine the effects on HDR of both the CTNNB1 and PRDM14 loci in iPSCs. Multiple small molecules include RS-1 (a stimulator of human homologous recombination protein RAD51), NU7441 (a DNA-PKcs inhibitor), SCR7 (a DNA ligase IV inhibitor), Brefeldin A, L755507, and

Nocodazole.

iPSCs were evenly split into eight wells after nucleofection with Cas9/sgRNA and the relevant double cut donor (pD-mNeonGreen-sg-HA300-300 bp or pD-GFP-sg-HA300-300 bp). DMSO control (0.1%), RS-1 (10 μM), Nu7441 (2 μM), SCR7 (1 μM), Brefeldin A (0.1 μM), L755507 (5 μM), Nocodazole (100 ng/mL), or Nu7441 (2 μM) and SCR7 (1 μM) were added in the wells for the first 24 h and then the medium was changed with fresh medium thereafter. Three days after nucleofection, cells were harvested for FACS analysis to determine the HDR efficiency in each condition.

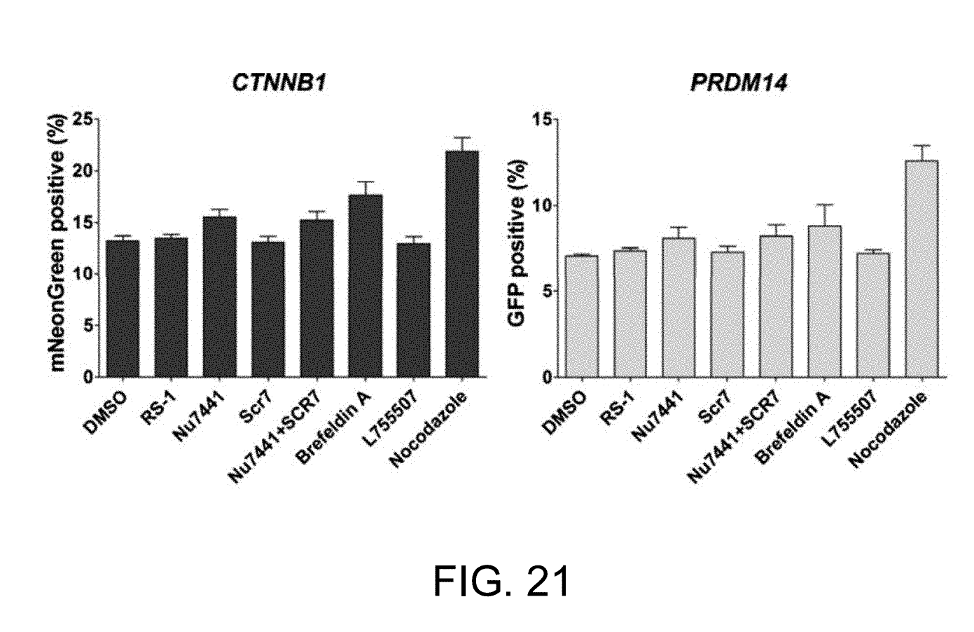

FIG. 21 shows the effects of small molecules on HDR efficiency at the CTNNB1 or PRDM14 locus in iPSCs. FACS. RS-1, SCR7, and L755507 did not show significant improvement in HDR efficiency at both the PRDM14 and CTNNB loci, while Nu7441 and Brefeldin A showed a less pronounced improvement only at the CTNNB locus (P<0.05). In contrast, treatment with Nocodazole, which synchronizes cell cycle at G2/M phase, increased HDR efficiency by ˜50% at PRDM14 and CTNNB loci (P<0.001) (FIG. 21).

[Effects of Small Molecules on HDR Efficiency at the CTNNB1 or PRDM14 Locus in the H1 ES Cell Line]

In this experiment, the H1 ES cells were evenly split into eight wells after nucleofection with Cas9/sgRNA and the relevant double cut donor (pD-mNeonGreen-sg-HA300-300 bp or pD-GFP-sg-HA300-300 bp). DMSO control (0.1%), RS-1 (10 μM), Nu7441 (2 μM), SCR7 (1 μM), Brefeldin A (0.1 μM), L755507 (5 μM), Nocodazole (100 ng/mL), or Nu7441 (2 μM) and SCR7 (1 μM) were added in the wells for the first 24 h and then the medium was changed with fresh medium thereafter. Three days after nucleofection, cells were harvested for FACS analysis to determine the HDR efficiency in each condition.

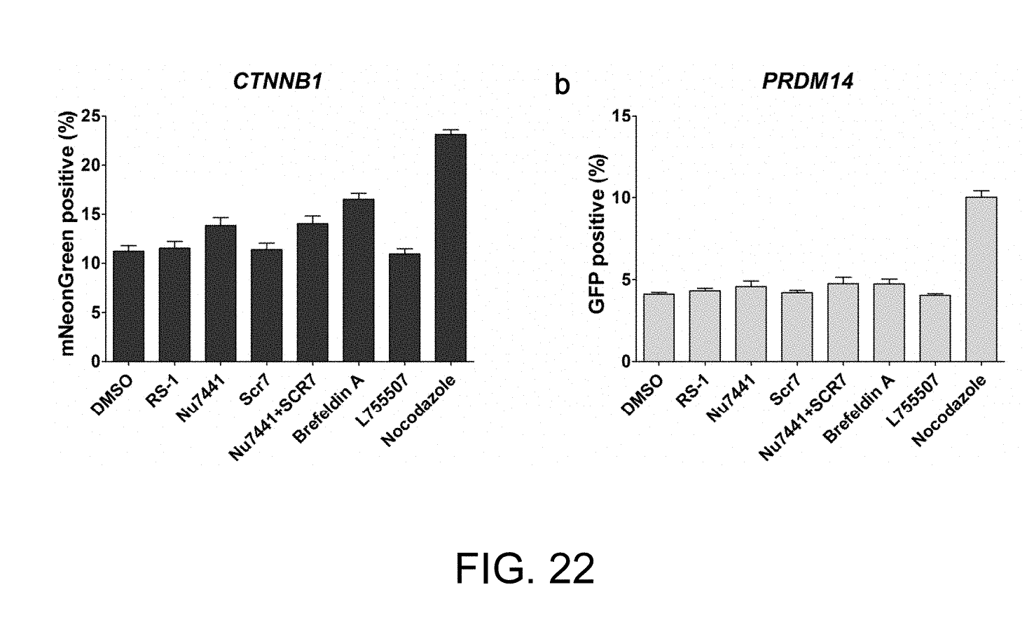

FIG. 22 shows the effects of small molecules on HDR efficiency at the CTNNB1 or PRDM14 locus in the H1 ES cells. Similar results were observed in H1 the human ES cell line. FACS. RS-1, SCR7, and L755507 did not show significant improvement in HDR efficiency at both the PRDM14 and CTNNB loci, while Nu7441 and Brefeldin A showed a less pronounced improvement only at the CTNNB locus. In contrast, treatment with Nocodazole, which synchronizes cell cycle at G2/M phase, increased HDR efficiency by ˜50% at PRDM14 and CTNNB loci (FIG. 22).

[Effects of RAD51, Ad4E1B-Eorf46, or CCND1 on HDR Efficiency at the CTNNB1 or PRDM14 Locus in iPSCs]

In this experiment, the extra factors (RAD51, CCND1, and Ad4E1B-E4orf6) were used to examine the effects on HDR of both the CTNNB1 and PRDM14 loci in iPSCs. RAD51 is a key factor in the homologous recombination pathway. Ad4E1B-E4orf6 was reported to considerably increase HDR by inhibiting NHEJ. CCND1, also known as cyclin D1, was reported to induces cell cycle transition from G0/G1 to S-phase.

RAD51, CCND1 and Ad4E1B-E4orf6 expression plasmid were constructed. Ad4E1B and E4orf6 were linked together using a ribosome-skipping E2A sequence. cDNAs for RAD51 and CCND1 were purchased from DNASU, and Ad4 E1B and E4orf6 were purchased from Addgene (Plasmid #64218 and 64222). The EF1 promoter was used to drive the expression of these genes and the Wpre-polyA cassette was tagged downstream of the stop codon to increase transgene expression levels. All plasmids were confirmed by sequencing.

The plasmid encoding RAD51, Ad4E1B-Eorf46, or CCND1 was co-transfected with Cas9, sgRNA, and donor plasmid (pD-mNeonGreen-sg-HA300-300 bp or pD-GFP-sg-HA300-300 bp). Three days after transfection, cells were harvested for FACS analysis to determine the HDR efficiency in each condition.

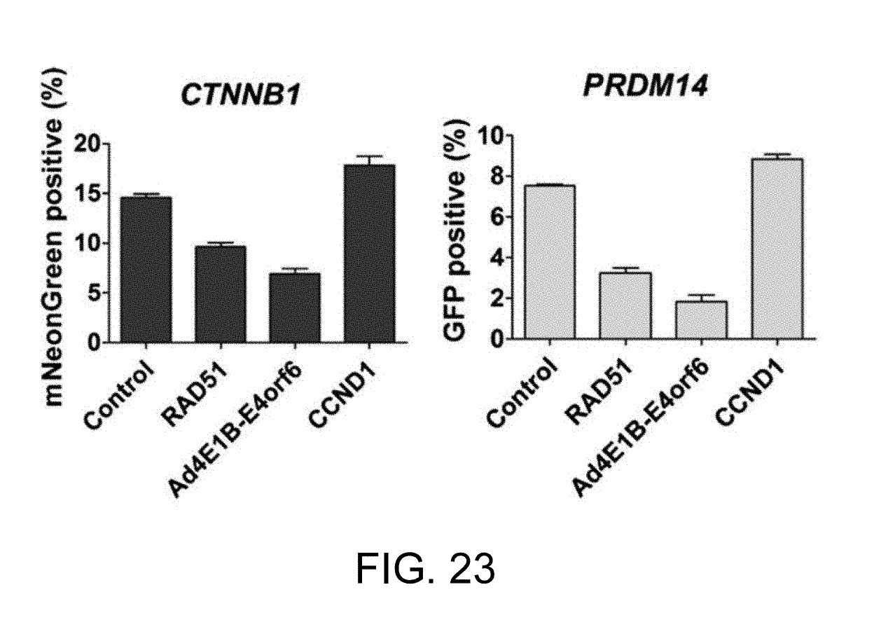

FIG. 23 shows the effects of RAD51, Ad4E1B-Eorf46, and CCND1 on HDR efficiency at the CTNNB1 or PRDM14 locus in iPSCs. RAD51 or Ad4E1B-E4orf6 significantly decreased HDR efficiency in our system (P<0.001), at the CTNNB1 and PRDM14 loci. In contrast, CCND1 showed a ˜20% improvement in HDR at both sites (FIG. 23).

[Effects of Nocodazole and CCND1 on HDR Efficiency at the CTNNB1 or PRDM14 Locus in iPSCs]

In this experiment, CCND1 and Nocodazole were used together to examine the effects on HDR of both the CTNNB1 and PRDM14 loci in iPSCs. The plasmid encoding CCND1 was co-transfected with Cas9, sgRNA, and donor plasmid (pD-mNeonGreen-sg-HA300-300 bp or pD-GFP-sg-HA300-300 bp). Nocodazole (100 ng/mL) was added into the medium at 0-24 h after transfection. Three days after incubation, cells were harvested for FACS analysis to determine the HDR efficiency in each condition.

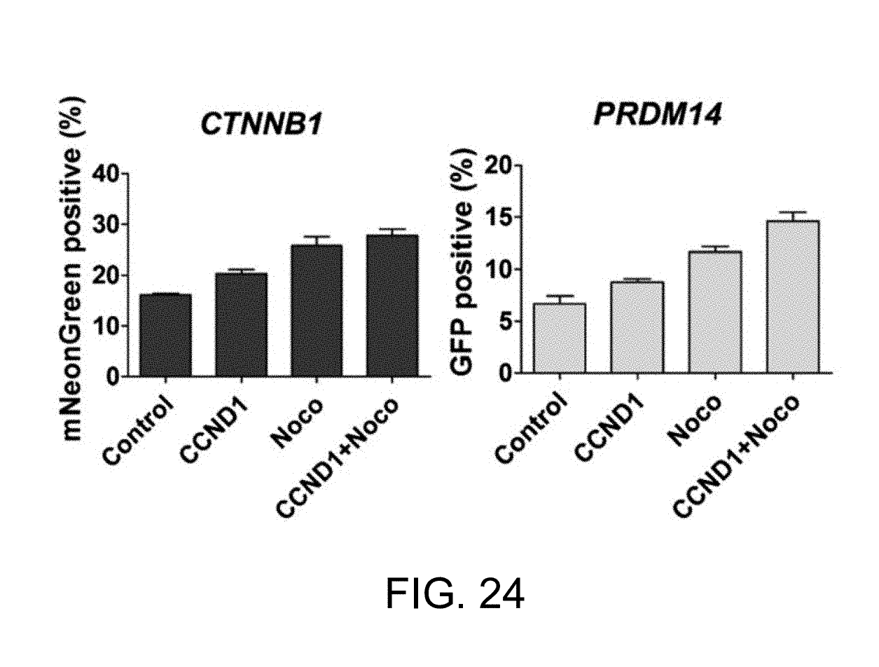

FIG. 24 shows the effects of Nocodazole and CCND1 on HDR efficiency at the CTNNB1 or PRDM14 locus in iPSCs. Combination of Nocodazole and CCND1 make an additive effect and increased HDR efficiency by 80˜100% at both loci (FIG. 24). This result may be explained by that the combined use of CCND1 and Nocodazole increases cells in S/G2/M phases during which HDR is efficient, while they considerably decrease cells in G0/G1 phase during which double-stranded DNA (dsDNA) breaks are predominated repaired by NHEJ. As such, Nocodazole and CCND1 have an additive effect on enhancing precise genome editing. The combined use of cell cycle regulators Nocodazole and CCND1 leads to an additional 100% increase in HDR efficiency.

[Effects of CCND1 on HDR Rate at the CTNNB1 Locus]

Human iPSCs were harvested at day 3 after cotransfection of Cas9, sgRNA, donor plasmid (pD-mNeonGreen-sg-RS1-0 bp-HA300-300 bp), and CCND1 plasmid for DNA extraction. The NHEJ insertion events in genome editing system were determined. The method of determining NHEJ-mediated and HDR-mediated knockin have been described above (see example 3). At least 30 colonies were picked for Sanger sequencing at both ends. As a control, cotransfection of Cas9, sgRNA, and donor plasmid (pD-mNeonGreen-sg-RS1-0 bp-HA300-300 bp) was performed.

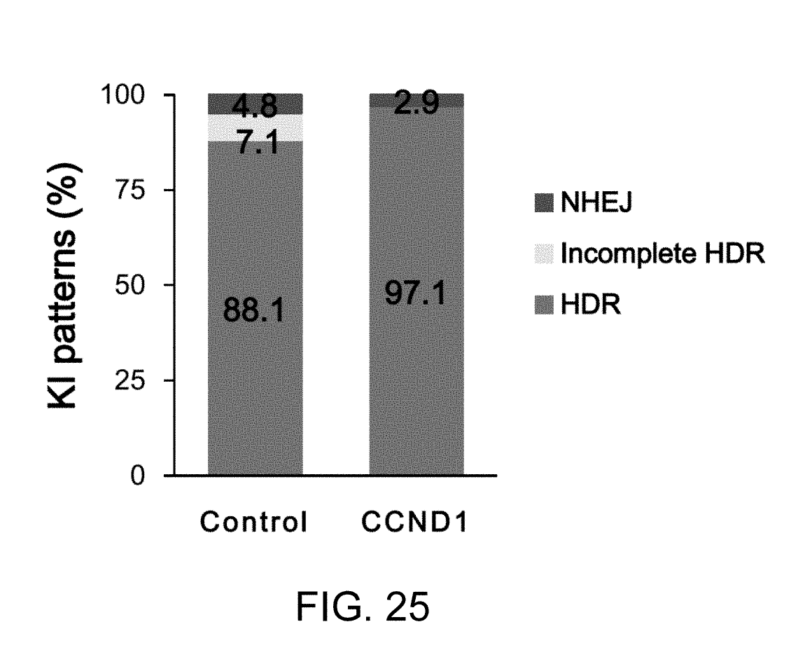

Table 4 shows a summary of Sanger sequencing results. FIG. 25 shows a distribution of different knockin patterns by CCND1. The HDR occurrence in bulk population increased from 77% to 88% (FIG. 25, Table 4). After co-transfection of iPSCs with CCND1 and HDR plasmids, the NHEJ events were decreased from 12% to 3% (FIG. 25). These results suggest that cell cycle regulators not only increase HDR but also suppress NHEJ.

TABLE 2

| | Number of clones | | | | | | | | | |
|---|---|---|---|---|---|---|---|---|---|---|
| | | | | | NHEJ | | | | | |
| | Complete | | Incomplete | | mediated | | | | | |
| | HDR | | HDR | | KI | | | | | |
| | No | with | 5-end | 3-end | mNeonGreen- | mNeonGreen- | Backbone- | Backbone- | Total | HDR |
| | mutation | mutation | NHEJ | NHEJ | forward | reverse | forward | reverse | number | (%) |
| control | 37 | 0 | 3 | 0 | 0 | 0 | 0 | 2 | 42 | 88.1 |
| CCND1 | 34 | 0 | 0 | 0 | 0 | 0 | 0 | 1 | 35 | 97.1 |

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

1. A method of inserting a donor sequence at a predetermined insertion site on a genome in an eukaryotic cell, comprising introducing a RNA-guided endonuclease, a guide RNA and a donor plasmid into the cell, and introducing a combination of cyclin D1 and Nocodazole,
wherein the donor plasmid comprises the donor sequence flanked with a 5′ homology arm and a 3′ homology arm, a 5′ flanking sequence upstream of the 5′ homology arm, and a 3′ flanking sequence downstream of the 3′ homology arm,
wherein the 5′ homology arm is homologous to a 5′ target sequence upstream of the insertion site on the genome and the 3′ homology arm is homologous to a 3′ target sequence downstream of the insertion site on the genome,
wherein the guide RNA recognizes the insertion site, the 5′ flanking sequence, and the 3′ flanking sequence,
wherein the RNA-guided endonuclease cleaves the genome at the insertion site,
wherein the RNA-guided endonuclease cleaves the donor plasmid at the 5′ flanking sequence and the 3′ flanking sequence to produce a linear nucleic acid, and
wherein the donor sequence is inserted in to the genome at the insertion site through homology-directed repair.

2. The method of claim 1, wherein the 5′ homology arm and the 3′ homology arm are at least about 50 bp in length, respectively.

3. The method of claim 1, wherein the 5′ homology arm and the 3′ homology arm range from about 50 bp to about 2000 bp in length, respectively.

4. The method of claim 1, wherein the 5′ target sequence and the 3′ target sequence are less than 200 bp away from the insertion site, respectively.

5. The method of claim 1, wherein the 5′ target sequence and the 3′ target sequence are separated by less than 200 bp.

6. (canceled)

7. (canceled)

8. The method of claim 1, wherein the combination of cyclin D1 and Nocodazole are introduced into the cell in the form of a protein, a mRNA, or a cDNA.

9. The method of claim 1, wherein the RNA-guided endonuclease is Cas9.

10. (canceled)

11. The method of claim 1, wherein the eukaryotic cell is a mammalian cell.

12. The method of claim 1, wherein the eukaryotic cell comprises a pluripotent stem cell or an adult stem cell.

13. A kit for inserting a donor sequence at a predetermined insertion site on a genome in an eukaryotic cell, comprising:
a RNA-guided endonuclease;
a guide RNA;
a donor plasmid;
cyclin D1; and
Nocodazole,
wherein the donor plasmid comprises the donor sequence flanked with a 5′ homology arm and a 3′ homology arm, a 5′ flanking sequence upstream of the 5′ homology arm, and a 3′ flanking sequence downstream of the 3′ homology arm,
wherein the 5′ homology arm is homologous to a 5′ target sequence upstream of the insertion site on the genome and the 3′ homology arm is homologous to a 3′ target sequence downstream of the insertion site on the genome,
wherein the guide RNA is able to recognize the insertion site, the 5′ flanking sequence, and the 3′ flanking sequence,
wherein the RNA-guided endonuclease is able to cleave the chromosome at the insertion site,
wherein the donor plasmid is cleaved at the 5′ flanking sequence and the 3′ flanking sequence within the cell to produce a linear nucleic acid.

14. The kit of claim 13, wherein the 5′ homology arm and the 3′ homology arm are at least about 50 bp in length, respectively.

15. The kit of claim 13, wherein the 5′ homology arm and the 3′ homology arm range from about 50 bp to about 2000 bp in length, respectively.

16. (canceled)

17. (canceled)

18. The kit of claim 13, wherein the RNA-guided endonuclease is Cas9.

19. (canceled)
